(19) United States
(12) Patent Application Publication
(10) Pub. No.: US 2025/0152015 A1
(43) Pub. Date: May 15, 2025

(54) METHOD AND APPARATUS FOR CARIES DETECTION

(71) Applicant: Align Technology, Inc., San Jose, CA (US)

(72) Inventors: Maayan MOSHE, Ramat Hasharon (IL); Moti BEN DOV, Tel Mond (IL); Shai FARKASH, Hod Hasharon (IL); Ran KATZ, Hod Hasharon (IL); Ran FEDER, Herzliya (IL); Hila SEGEV, Ramat Ishay (IL); Roie COHEN, Givatayim (IL); Uriel BARRON, Tel Aviv (IL)

(21) Appl. No.: 18/943,878

(22) Filed: Nov. 11, 2024

Related U.S. Application Data
(60) Provisional application No. 63/598,068, filed on Nov. 10, 2023.

(51) Int. Cl. A61B 5/00; A61B 1/00; A61B 1/24; G16H 30/40

(52) U.S. Cl. CPC A61B 5/0088; A61B 1/000096; A61B 1/00172; A61B 1/24; G16H 30/40

(57) ABSTRACT

Methods and apparatuses for dental caries detection and communication. These methods and apparatuses may be used in real time, as part of an intraoral scanning system, or at any point following intraoral scanning. These methods and apparatuses may use a plurality of different images of the teeth, each having two or more fields (e.g., wavelengths such as visible light, near-infrared, fluorescent, etc.), and may use a trained pattern matching agent to detect possible caries centers from each image, then may project some or all of the possible caries centers onto a 3D model of the teeth to identify consensus caries and caries centers.

### CLAIM OF PRIORITY

This patent application claims priority to U.S. provisional patent application No. 63/598,068, titled “METHOD AND APPARATUS FOR CARIES DETECTION,” and filed on Nov. 10, 2023, herein incorporated by reference in its entirety.

### BACKGROUND

Dental caries may be characterized by tooth demineralization leading to an increase in the porosity of the enamel surface. Leaving these lesions untreated may potentially lead to cavities reaching the dentine and pulp and perhaps eventually causing tooth loss. Occlusal surfaces (bite surfaces) and interproximal regions (between the teeth) are among the most susceptible sites of demineralization due to acid attack from bacterial by-products in the biofilm.

Traditional methods for caries detection include visual examination and tactile probing with a sharp dental exploration tool, often assisted by radiographic (x-ray) imaging. However, detection using these methods is subjective and may miss caries, particularly at early stages. Although it has been proposed to use techniques such as light absorption, scattering, transmission, reflection and/or fluorescence, and near-IR light to detect caries, thus far these techniques have proven difficult and computationally expensive. Such images have proven difficult to assess and to provide reliable indicators of caries.

Thus, there is a need for detection of lesions, including at early stages, in order to treat the caries, e.g., to inhibit or reverse the demineralization.

### SUMMARY OF THE DISCLOSURE

Described herein are methods and apparatuses for dental caries detection and communication. These methods and apparatuses may be used in real time, e.g., as part of an intraoral scanning system, or at any point following intraoral scanning. These methods and apparatuses may use a plurality of different images of the teeth, each having two or more fields (e.g., wavelengths such as visible light, near-infrared, fluorescent, etc.), and particularly multiple images of the same region of the teeth, and may use a trained pattern matching agent (e.g., a trained machine learning agent) to detect possible caries centers from each image, then may project some or all of the possible caries centers onto a 3D model of the teeth to identify consensus caries and caries centers.

As mentioned, any of these methods and apparatuses (e.g., devices, systems, etc. including software, hardware and/or firmware) may use a trained machine learning agent. In particular, described herein are methods and apparatuses for detection of caries from intraoral scanner data, for example, after image positioning and/or the formation of a three-dimensional (3D) surface model using the same images, e.g., intraoral scan images, that are used to generate the 3D model. The identified or suspected caries may be displayed on the 3D surface model of the teeth.

For example, described herein are methods, e.g., methods for detecting and/or displaying caries, that may include: receiving or collecting a plurality of two-dimensional (2D) intraoral scanner images of a patient's teeth, wherein each image comprises two or more channels, including a near-infrared (near-IR) and a visible light channel; identifying, for each image of the plurality of 2D intraoral scanner images, a caries center for any carries in the images using a trained pattern matching agent that is trained to use the two or more channels of each image; projecting the caries center for any carries identified in each image onto a three-dimensional (3D) model of the patient's teeth, wherein the 3D model of the patient's teeth is generated from the plurality of 2D intraoral scanner images; determining a consensus caries center for each of one or more caries based on the projected detected caries; and outputting an indicator of one or more caries based on the consensus caries centers.

In any of these methods identifying the caries center for any carries in the images may include identifying caries centers and caries boundaries from each image. Caries boundaries may be determined using the trained pattern-matching agent for each image and may be marked on the image or referenced from the image. Coordinates for the caries boundary and/or caries center may be determined using the camera position from when the image was scanned. Similarly, the trained pattern matching agent may indicate the carries center, or a caries center identification module may be used once the pattern matching agent has identified the possible caries boundary from the image. The caries center may be the approximate midpoint (or region) from the edges of the caries boundary. In some cases, the caries center may be the center of the region of the most severe caries.

In general, the methods and apparatuses described herein may determine the caries centers and/or the boundary of the caries using a plurality (e.g., 5 or more 10 or more, 15 or more, 20 or more, 30 or more, 40 or more, 50 or more, 60 or more, 70 or more, 80 or more, 90 or more, etc.) images, and their corresponding multiple channels, that show the same or overlapping regions of a tooth, and in some cases, overlapping regions of the tooth that include the caries, and in particular the caries center(s) on the tooth, from multiple different camera angles. Some angles, particularly more lingual and buccal angles, may be difficult to identify caries and particularly interproximal caries. Both false positives and false negatives for caries identification may be a result of the angles (and resulting shadows, irregular tooth shapes, etc.). The methods and apparatuses described herein may us a relatively large number of images of approximately the same caries-containing regions (based on the putative caries centers) that may be used to determine (e.g., by a weighted or unweighted vote) a confidence level for a particular caries or corresponding caries center; this confidence level (confidence score) may be compared to a threshold to determine if a potential (portative) caries, and corresponding potential caries center, is considered a likely (or actual) caries and/or caries center. The confidence level may be based on the percentage and/or number of images identified as having a potential caries from the trained pattern matching agent as compared with the total number of image of the same, overlapping caries region and/or caries center. The confidence level may be based in part on weighting the contribution of individual images based, e.g., on a confidence weight that may be determined by the trained pattern matching agent and/or based on one or more features in the image and/or based on the camera angle (e.g., angles taken from occlusal direction may be weighted more than angles taken from more buccal and/or lingual directions). In some examples, the confidence level (confidence score) for a particular caries center may be based on an average or sum of the individual confidence weights for the plurality of images of the same tooth region including the caries center(s) taken from different camera angles.

For example, identifying the caries center for any carries in the images may include identifying caries centers from at least x images (where x is 5, 10, 15, 20, 25, 30, 35, 40, 45, 50, 55, 60, 65, 70, 75, 80, 85, 90, 95, 100, 110, 120, 130, 140, 150, 160, 170, 180, 200, etc.) of the same region of a tooth, e.g., including the caries center(s), taken with different camera angles. The overlapping regions of the tooth may be determined based on proximity of the caries centers identified by the trained pattern matching agent, or based on overlap between the caries boundaries. In some examples, the overlapping regions may be based on the 3D model of the teeth. Thus, the method or apparatus may identify subsets of images from the plurality of scan images that cover similar or overlapping regions of the surfaces of the teeth. In some cases, these subsets may be determined based on overlapping or similar regions in which at least one possible caries center was identified.

Thus, in any of these methods an apparatuses, identifying the caries center for any carries in the images may further comprise confirming that the caries center corresponds to a carries based on a comparison of a subset of images of the plurality of 2D intraoral scanner images having an overlapping view of a region of a tooth including the caries center, wherein each of the plurality of images is taken from a different camera angle. The subset of images of the plurality of 2D intraoral scanner images having the overlapping view of the region of the tooth including the caries center may comprises at least x images (e.g., where x is 1 or more, e.g., 3 or more, 4 or more, 5 or more, 6 or more, 7 or more, 8 or more, 9 or more, 10 or more, 15 or more, 20 or more, 30 or more, etc.). The step of confirming the caries (and/or confirming the caries center) may include applying a threshold based on a number and/or a caries confidence score for each image of the subset of images.

Although each image (and corresponding channels for each image) may be analyzed by the trained pattern matching agent to identify one or more caries region and/or caries centers. Because each image may be taken from a slightly different camera position, the location of the actual caries center for each caries (confirmed or possible caries) may be slightly different. Thus, these methods and apparatuses may determine a consensus caries center and/or caries boundary from the plurality of images. In some examples, determining the consensus caries center comprises aggregating the projected caries centers into groups of projected caries centers, by applying a distance threshold to the projected caries.

The methods and apparatuses described herein may output the caries, e.g., caries centers and/or caries boundaries, and/or information, such as scores, about the caries. Any appropriate output may be used. In some cases, outputting may include displaying, storing and/or transmitting either or both a labeled 3D model of the teeth (labeled or otherwise marked to indicate the presence of caries) and/or a caries data structure. For example, any of these methods may include outputting a caries detection 3D model of the patient's teeth including the consensus projected carries centers distributed on the caries detection 3D model. Outputting may include outputting a caries data structure that includes one or more or: an indicator of the position of the caries in the teeth (e.g., narrative information about which teeth, by tooth number and/or name, the caries is present on, a position of the caries center and/or boundary), an indicator of the severity of the caries (e.g., a clinical severities score), an indicator of the area and/or volume of the caries, etc.

In some examples, when caries detection is performed in real time (e.g., as part of or in conjunction with an intraoral scanner), the apparatus or method may include indicating to a user and/or guiding the user to rescan or further scan a region having one or more caries.

In general, any of these methods and apparatuses may include segmenting the 3D model of the patient's teeth before or after projecting the detected caries center to the 3D model. The 2D images and/or the 3D model may be segmented. Segmentation may help distinguish if a caries is on a particular tooth or corresponding to multiple different caries (e.g., interproximal caries).

In general, receiving or collecting the plurality of 2D intraoral scanner images of the patient's teeth may include receiving the plurality of 2D intraoral scanner images from an intraoral scanner data set. For example, receiving or collecting the plurality of 2D intraoral scanner images of the patient's teeth may include receiving a plurality of 2D image comprising four or more channels including a near-infrared (near-IR), a red light, a green light, and a blue light channel. Identifying the caries center for any carries in the images using the trained pattern matching agent may include identifying caries centers for each of at x images (e.g., where x is 2 or more, e.g., 3 or more, 4 or more, 5 or more, 6 or more, 7 or more, 8 or more 9 or more, 10 or more, 15 or more, 20 or more, 30 or more, 40 or more, 50 or more, 60 or more, 70 or more, 80 or more, 90 or more 100 or more, 110 or more, 120 or more, 150 or more, 200 or more, etc.) having overlapping tooth regions of the plurality of 2D intraoral scanner images.

In any of these methods, determining the consensus caries center for each of one or more caries based on the projected detected caries may include generating a caries data structure including coordinates of each of the consensus caries center, a severity of the caries, a reference to a representative image for each caries. For example, any of these methods may include determining a severity classification for each caries associated with the consensus caries centers.

For example, a method as described herein (e.g., for identifying caries) may include: receiving or collecting a plurality of two-dimensional (2D) intraoral scanner images of a patient's teeth, wherein each image comprises two or more channels, including a near-infrared (near-IR) and a visible light channel; identifying, for each image of the plurality of 2D intraoral scanner images, a caries center for any carries in the images using a trained pattern matching agent that is trained to use the two or more channels of each image; confirming that the caries center corresponds to a carries based on a comparison of a subset of images of the plurality of 2D intraoral scanner images having an overlapping view of a region of a tooth including the caries center, wherein each of the plurality of images is taken from a different camera angle; projecting the confirmed caries centers onto a three-dimensional model of the patient's teeth; aggregating the projected caries centers to determine a consensus caries center for each of one or more caries based on the projected confirmed caries centers; and outputting an indicator of one or more caries based on the consensus caries centers.

The methods described herein provide a dramatic improvement over prior attempts to identify caries from images of the teeth, even prior attempts using different wavelengths of light. The use of additional, unconventional steps including identifying an indicator associated with the caries, such as a caries boundary and/or caries center, confirming putative caries based on voting or weighted voting from a plurality of images of the same region taken from different camera angles and/or projecting caries onto a 3D model of the teeth, and particularly a segmented model, may provide dramatically more accurate and time and cost-effective techniques for identifying caries, while minimizing false negatives as well as false positives.

Also described herein are systems for performing any of these methods and/or software for performing any of these methods. In general, systems that are configured to perform these methods may be configured as (or as part of) an intraoral scanner. For example, a system may include: one or more processors; and a memory coupled to the one or more processors, the memory storing computer-program instructions, that, when executed by the one or more processors, perform a method comprising: receiving or collecting a plurality of two-dimensional (2D) intraoral scanner images of a patient's teeth, wherein each image comprises two or more channels, including a near-infrared (near-IR) and a visible light channel; identifying, for each image of the plurality of 2D intraoral scanner images, a caries center for any carries in the images using a trained pattern matching agent that is trained to use the two or more channels of each image; projecting the caries center for any carries identified in each image onto a three-dimensional (3D) model of the patient's teeth, wherein the 3D model of the patient's teeth is generated from the plurality of 2D intraoral scanner images; determining a consensus caries center for each of one or more caries based on the projected detected caries; and outputting an indicator of one or more caries based on the consensus caries centers.

The system may include an intraoral scanner. The one or more processors may be part of (or alternatively separate from) the intraoral scanner). The memory storing the instructions for performing the method may be part of (or alternatively separate from) the intraoral scanner. For example, outputting may include outputting in real time as the intraoral scanner is scanned over the patient's teeth. The system may be configured to perform any of the steps described above. For example, the system may be configured so that identifying the caries center for any carries in the images comprises identifying caries centers and caries boundaries from each image.

For example, a system may include: an intraoral scanner; one or more processors; and a memory coupled to the one or more processors, the memory storing computer-program instructions, that, when executed by the one or more processors, perform a method comprising: receiving or collecting a plurality of two-dimensional (2D) intraoral scanner images of a patient's teeth, wherein each image comprises two or more channels, including a near-infrared (near-IR) and a visible light channel; identifying, for each image of the plurality of 2D intraoral scanner images, a caries center for any carries in the images using a trained pattern matching agent that is trained to use the two or more channels of each image; confirming that the caries center corresponds to a carries based on a comparison of a subset of images of the plurality of 2D intraoral scanner images having an overlapping view of a region of a tooth including the caries center, wherein each of the plurality of images is taken from a different camera angle; projecting the confirmed caries centers onto a three-dimensional model of the patient's teeth; aggregating the projected caries centers to determine a consensus caries center for each of one or more caries based on the projected confirmed caries centers; and outputting an indicator of one or more caries based on the consensus caries centers.

The software performing any of these methods may be local (e.g., part of the intraoral scanner) and/or remote, including accessing from a remote site. In practice, some of the functions may be performed locally, while some may be remote (e.g., the trained pattern matching agent may be remote, and/or local, etc.).

In some examples, the method or apparatus may receive at least 2 channels, at least three channels, at least four channels, etc. In some examples, each image may include four channels: one near-IR channel, one red channel, one blue channel and one green channel. The trained pattern matching agent (trained AI network) may identify possible caries indicators (e.g., caries centers and/or caries boundaries) from each image using the plurality of different channels. Each image and/or each channel may include the associated camera angles from which the images (and/or channel) was taken. In general, the channels forming each “image” may be taken from similar, though slightly shifted, camera positions, as the scanner taking the image may be moving (scanning) over the teeth. In some cases, the change in position may be very small (e.g., less than a few micrometer, less than 5 μm, less than 10 μm, less than 20 μm, less than 50 μm, less than 80 μm, less than 100 μm, less than 150 μm, etc.). The caries centers (which may be caries center points or regions) corresponding to each potential caries in each of a plurality of images may be used to detect, processes and/or confirm the presence of actual caries.

In some cases, more than one trained pattern recognition agent may be used in the methods and apparatuses described herein. For example, in some cases, an adversarial set of trained pattern recognition agents may be used to identify caries regions and/or caries centers; a first pattern recognition agent may be trained to identify region corresponding to caries and/or caries centers and a second pattern recognition agent may be trained to recognize regions that are not caries, and the pair may be used together to remove or reduce false positives.

In general, the methods and apparatuses described herein may combine, e.g., aggregate, caries regions (e.g., caries borders) and/or caries centers, and in particular may determine a consensus caries region/border and/or caries center from a plurality of images of the same general region of the tooth in order to get a final caries region/border and/or caries center, which may provide a single 3D detection flag. As described above, one or more threshold may be used to determine if a caries is likely to be a real caries, e.g., if the aggregated detection region (e.g., a score for the aggregated region) is above a threshold value, as described.

In some examples, the methods described herein may generate one or more groups or subset of images by grouping images for which the dental caries centers are within a minimum distance of each other (e.g., requiring a minimal difference between caries sections based which may be estimated from the image and the camera vector corresponding to the images). In some cases, the method or apparatus may set a minimum number of images per group or subset and may prioritize images in the group that are closest together for central region to give them highest confidence value. The methods or apparatuses may then identify one or more images with the highest confidence value as “best” in each group.

In general, the methods and apparatuses described herein may segment the images and/or 3D model or may use a segmented image or 3D model of the teeth to differentiate between caries on different teeth, even when the teeth are close together. For example, any of these methods may segment or apply segmentation of teeth and tooth numbering at each image, and in particular images for which a caries was detected. Caries centers that are on different teeth, based on the segmentation may be kept as separate caries and caries centers.

As mentioned, any appropriate output may be used, including, but not limited to, outputting a final 3D surface image that may be annotated with one or more indicators of dental caries determined as described herein. The output may be surface 3D image with detected caries to user interface for display. In some cases, an indicator of the caries (e.g., caries centers, a marker, etc.) may be displayed on the 3D model and the user may select one or more of these indicators to show additional detail, including textual detail, such as a tooth number/location, the severity of the caries, one or more representative images, etc. The output may also be graphical, such as markings of the caries, caries outline, caries center, etc. on the 3D model.

Also described herein are methods and apparatuses for determining the severity of a dental caries. For example, any of these methods may include identifying a characteristic of the dental caries, and in particular a caries center, a described above, e.g., using a trained pattern matching (e.g., machine learning) agent. The method may then include identifying actual images (and/or generating synthetic images) of the region around the center on surface of teeth and/or within volume using one or more channels, such as the NIR image channel, to form map on surface and/or within a 3D volume of the teeth. The method or apparatus may then divide the surrounding region up into sub-regions and generate images (actual or synthetic) for each, then analyze the sub-region within each image to determine if the sub-region includes one or more caries. For each image around the identified caries center(s), the method and apparatus may determine a caries boundary and may optionally project this region boundary onto the 3D model of the teeth. In some examples, for each sub-region (and/or for the caries center) the method or apparatus may determine a “score” related to how likely that an identified caries (e.g., caries region or associated caries center) is an actual caries. Any of the methods and apparatuses described herein may also or additionally include generating a caries severity map (e.g., classifying caries severity using a standard scale, such as an E1/E2 scale, et.).

In some examples, the method or apparatus may identify the one or more caries and may present them to a user, such as a dental technician, patient, etc. In some examples, the user may select or adjust a display threshold to display caries (or caries centers) having a confidence level above the display threshold. The apparatus or method may then display, e.g., one or more images of the caries from the 2D images and/or on the 3D model; in some cases, display(s) may include a selectable link associated with each marked caries that may be selected to provide additional information about the caries. The output may also allow interaction by the user, for example, allowing the user to tag or mark or select one or more caries (caries centers, etc.) for follow-up, such as treatment, and/or for tracking over time, etc.

Also described herein are methods for updating a patient record using the caries detection methods and/or apparatuses described herein. For example, any of these methods may provide an output to update a patient record (e.g., dental record, medical record, etc.). An output of the caries detection, including automatic caries detection, using any of the methods described herein may include outputting a list of the detected caries above a user-defined or preset threshold. The list may be a textural listing and/or description of the caries. Alternatively, or in addition, the output may include one or more image (e.g., a representative image) of the caries. A textual description of the caries may include a textural description of the location (e.g., which tooth the caries is on), classification of the caries, size of the caries, etc.), a descriptor of severity, etc.

Also described herein are methods and apparatuses for tracking caries over time. For example, any of these methods may be used for monitoring one or more characteristics of one or more caries over time, including the size (surface area, volume, etc.) of the caries, severity (e.g., severity score, etc.), caries center, etc. over time between one or more scans of the patient's teeth. This may be referred to herein as longitudinal tracking.

Also described herein are methods and apparatuses for displaying images, including, but not limited to near-IR images, which may best show a caries, should caries be present on the teeth. These methods and apparatuses may be used to identify one or more images, and preferably a small subset of images, from an intraoral scan that are most likely to include caries. The resulting selected images may be examined to determine if caries are present, either manually or automatically. In general, these methods and apparatuses may identify a preselected subset of images that are more likely to have visible caries, based on grading the images to identify those images in which the tooth orientation and imaging properties (e.g., light and camera properties). The resulting subset may reduce the total number of images from an intraoral scan (e.g., total number of near-IR images) from a relatively large number of images (which may be greater than 500, greater than 750, greater than 1000, etc.) to a fraction of this amount, e.g., 25% or less, 20% or less, 15% or less, 10% or less, 9% or less, 8% or less, 7% or less, 6% or less, 5% or less, 4% or less, 3% or less, 2% or less, 1% or less, etc. This may save the user a time in reviewing the intraoral scan. For example, these methods and apparatuses may provide a score (e.g., a numeric/quantitative score and/or a qualitative score) of each of the images, and/or each of the near-IR images, and/or each of the visible light images, etc. The method or apparatus may then display a subset of the scored images having a score that is above a threshold value and/or may display (or transfer to a subset) a fixed number of images having the highest score. In general, the score may provide a ranking, which may be arranged, for example, in highest value to lowest value, where the magnitude of the score value represents the likelihood that the particular scored image shows a caries. As mentioned, caries may not actually be present in all or any of these images. The technique and/or system is simply scores image that, if a caries is present, are likely to show it.

Any of the intraoral scanners described herein may be configured to determine which images from an intraoral scan are likely to include a caries. In particular, these apparatuses (e.g., systems or device, including software, firmware and/or hardware) may be configured to review images of an intraoral scan, e.g., all or some of the images, including all or some of the near-IR images, by identifying one or more reference points on a tooth in the image(s), and determining for the one or more reference points, if the imaging properties of the intraoral scanner, such as the camera properties and/or lighting properties, as well as the surface properties of the tooth at the reference point are such that the image is likely to show a caries.

For example, an intraoral scanning system may include: a wand comprising a near-infrared (near-IR) light source and one or more cameras; one or more processors; and a memory coupled to the one or more processors, the memory storing computer-program instructions, that, when executed by the one or more processors, cause the processor to: access a plurality of near-infrared (near-IR) images of a subject's teeth taken by the wand; score each near-IR image of the plurality of near-IR images to provide on a likelihood that a caries is visible in the near-IR image based on one or more features describing a relationship between one or more reference points in an interproximal region of a tooth in the near-IR image and one or more camera parameters of a camera taking the near-IR image by: identifying, for each image of the plurality of near-IR images, the one or more reference points, a normal to the reference point relative to the tooth, and a camera angle of the camera taking the near-IR images, and estimating an angle between the normal and the camera angle; and display near-IR images from the plurality of near-IR images based on the scores.

In general, these methods and apparatuses may be configured so that the intraoral scanning system includes a wand (having one or more cameras, one or more light sources, etc.). The one or more cameras may include a camera (e.g., CCD camera) and optics (e.g., beam splitters, filters, gratings, polarizers, wavelength selective quarter waveplate, etc.) that may be configured to detect near-IR, fluorescence, visible light (e.g., white light), single-wavelength light (e.g., red, blue, green, etc.). Any of these apparatuses may include one or more light sources to emit one or more wavelengths appropriate for near-IR, white light, etc. Any of these apparatuses may be configured to emit light that results in fluorescence in the teeth or other oral targets. In some cases, the intraoral scanner includes a housing (e.g., base, cabinet, etc.) that encloses all or some of one or more processors and associated circuitry, including memory. For example, these apparatuses may include a base enclosing the one or more processors and the memory. The intraoral scanning systems described herein may include one or more visible light sources on the wand (e.g., near-IR light source, such as an LED, white light source, such as an LED, laser, etc.).

In any of these apparatuses, the instructions cause the one or more processors to score near-IR images having smaller angles between the normal and a camera angle higher than images having larger angles between the normal and the camera angle. An intraoral scanning system may execute instructions (e.g., software and/or firmware) from the memory that cause the one or more processors of the intraoral scanner to identify, for each image of the plurality of near-IR images (or a subset of these images), one or more parameters that may be used to directly score each image or that may be used as an input into a trained machine learning agent (e.g., as a classifier) that may score each image. In general these one or more parameters may be based on one or more points in/on the teeth, referred to herein as reference point(s), and/or may be based on a characteristic of the intraoral scanner, and in particular, the wand, such as the light source or light sources (near-IR and/or visible light, fluorescence source, etc.) and/or camera. For example, these apparatuses and methods may determine a light source vector for each of one or more light sources, a vector between the reference point and the camera, a tooth axis for the tooth, the angle between the tooth axis and the camera angle, a luminosity of each of the one or more light sources, a refraction of the camera angle to the reference point, distances between the camera and the reference point, distances between the camera and the light sources, distance between the camera and the one or more light sources.

Any of the apparatuses and methods described herein may also identify the one or more reference points. For example, an intraoral scanning system may be configured to score the images using a trained machine-learning (ML) agent to score based on all or some of these parameters, which may themselves be features or may be used to determine features such as, but not limited to: a function of an angle between the camera and a normal at the reference point (e.g., Cosine of the angle between camera and normal at reference point), a measure the z distance of a vector between the reference point and the camera (e.g., Z absolute value of reference point to camera vector, a camera to reference point distance in mm), a function of an angle between the camera direction and the reference point (e.g., cosine of the angle between camera direction and reference point), a magnitude of a camera direction vector (e.g., absolute of z value of camera direction vector), a function of an angle between the camera refraction and a tooth axis of the tooth (e.g., cosine of the angle between camera refraction and tooth axis), a magnitude of a light source direction vector (absolute z value of LED direction vector), a function of an angle between a light source direction vector and the normal at the reference point (e.g., cosine of the angle between led1 and normal), a function of an angle between the first light source direction vector and the reference point (e.g., Cosine of the angle between led1 direction and reference point), a distance between the first light source direction and the reference point (e.g., Distance between LED1 location and reference point in mm), a distance between the first light source and the camera (e.g., Distance between led1 location and camera in mm), a luminosity of the first light source (e.g., LED1 luminosity), a measure of the z-distance of a vector between the reference point and the first light (e.g., Absolute of z value of reference point to led1 vector), a function of an angle between a second light source direction vector and the normal at the reference point (e.g., Cosine of the angle between led2 direction and normal), a luminosity of the second light source (e.g., LED2 luminosity), a z-magnitude of the vector between the reference point and the second light source (e.g., Absolute of z value of reference point to LED2 vector), a function of an angle between the direction of the second light source and the reference point (e.g., Cosine of the angle between led2 direction and reference point), a distance between the second light source and the reference point (e.g., Distance between led2 location and reference point in mm), and a distance between the second light source and the camera (e.g., Distance between LED2 location and camera in mm).

Any of these apparatuses (e.g., intraoral scanning systems) may be configured to score the images by identifying the one or more reference points on the interproximal region of each tooth by segmenting the plurality of 2D images to identify a tooth surface, identifying the interproximal region, and selecting the one or more reference points from within the interproximal region.

The apparatus may be configured so that they score each image of the plurality of near-IR images by generating a score between 0-1. In some cases, the apparatus may be configured to associate the score with each near-IR image in a database along with the near-IR images. In some cases, a data set that references (e.g., points to, addresses, etc.) the corresponding image may include the score. This data set (data structure) may be any appropriate data structure and may be accessed by the apparatus to search, from one or more sub-sets and be used as a reference for the intraoral scan. In some cases, the score may be appended (e.g., included with) to each image(s), e.g., as metadata.

In general, these apparatuses (e.g., systems) may be configured to select near-IR images from the plurality of near-IR images for display having a score that is more than or equal to a threshold. The one or more processors to rank the near-IR images from the plurality of near-IR images by score and display a predefined number of near-IR images having the highest ranking scores.

Also described herein are methods, including methods of displaying images likely to have carries from an intraoral scan, that include: receiving or accessing a plurality of near-infrared (near-IR) images of a subject's teeth; scoring each near-IR image of the plurality of near-IR images to provide on a likelihood that a caries is visible in the near-IR image based on one or more features describing a relationship between one or more reference points in an interproximal region of a tooth in the near-IR image and one or more camera parameters of a camera taking the near-IR image by: identifying, for each image of the plurality of near-IR images, the one or more reference points, a normal to the reference point relative to the tooth, and a camera angle of the camera taking the near-IR images, and estimating an angle between the normal and the camera angle; and displaying near-IR images from the plurality of near-IR images based on the scores. As mentioned, the scoring may include locally scoring the images in a processor of an intraoral scanner that took the plurality of near-IR images.

In some cases, scoring may be based entirely on the shape (e.g., angel) of the tooth and the imaging (e.g., camera, light source(s), etc.) properties. For example, scoring the images may include scoring images having smaller angles between the normal and a camera angle higher than images having larger angles between the normal and the camera angle. Alternatively, or additionally, scoring may be based on a variety of different properties between the tooth surface, camera(s) and light(s) that may provide multi-dimensional properties which may correlate with the likelihood of caries being present in a particular image. For example, scoring each near-IR image of the plurality of near-IR images may further comprises identifying, for each image of the plurality of near-IR images, one or more of: a light source vector for each of one or more light sources, vector between the reference point and the camera, a tooth axis for the tooth, the angle between the tooth axis and the camera angle, a luminosity of each of the one or more light sources, a refraction of the camera angle to the reference point, distances between the camera and the reference point, distances between the camera and the light sources, distance between the camera and the one or more light sources. As already mentioned above in the context of the apparatus, scoring may comprise using a trained machine-learning (ML) agent (e.g., network) to score based on the one or more features; the trained ML agent may receive as input one or more features comprises one or more of: a function of an angle between the camera and a normal at the reference point, a measure the z distance of a vector between the reference point and the camera, a function of an angle between the camera direction and the reference point, a magnitude of a camera direction vector, a function of an angle between the camera refraction and a tooth axis of the tooth, a magnitude of a light source direction vector, a function of an angle between a light source direction vector and the normal at the reference point, a function of an angle between the first light source direction vector and the reference point, a distance between the first light source direction and the reference point, a distance between the first light source and the camera, a luminosity of the first light source, a measure of the z-distance of a vector between the reference point and the first light, a function of an angle between a second light source direction vector and the normal at the reference point, a luminosity of the second light source, a z-magnitude of the vector between the reference point and the second light source, a function of an angle between the direction of the second light source and the reference point, a distance between the second light source and the reference point, and a distance between the second light source and the camera.

In any of these apparatuses, scoring may comprise identifying the one or more reference points on the interproximal region of each tooth by segmenting the plurality of 2D images to identify a tooth surface, identifying the interproximal region, and selecting the one or more reference points from within the interproximal region. As mentioned, scoring each image of the plurality of near-IR images may include generating a score between 0-1. Scoring may comprise associating a score with each near-IR image in a database including the near-IR images.

The resulting scores may be used to determine which images to display, or to prepare for display. In some cases, the scores may be used to form a sub-set of images for display, or for directly displaying. The images may be displayed together (e.g., adjacent, including side-by-side, in different windows/frames, etc.) and/or as a series, including as a movie. A user may use a user interface to view and manipulate the images, including switching between the views, selecting/storing views, etc. In some cases, the sub-set of views (or a further subset of them) may be stored in a patient's record (e.g., dental chart, etc.). In any of these methods, displaying may comprise selecting near-IR images from the plurality of near-IR images for display having a score that is more than or equal to a threshold. For example, displaying may include ranking the near-IR images from the plurality of near-IR images by score and displaying a predefined number of near-IR images having the highest ranking scores.

In some examples, a method of displaying images likely to have carries from an intraoral scan may include: receiving or accessing a plurality of near-infrared (near-IR) images of a subject's teeth; scoring each image of the plurality of near-IR images to provide on a likelihood that a caries is visible in the image, wherein the score is based on one or more features describing a relationship between one or more reference points in an interproximal region of a tooth in the near-IR image and one or more camera parameters of a camera taking the near-IR image; selecting a subset of the plurality of near-IR images based on the scores; and displaying the near-IR images of the subset.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 schematically illustrates an example of an apparatus for detecting caries as described herein.

FIG. 2 illustrates an example of a method of detecting caries as described herein.

FIGS. 3A-3F illustrate an example of caries detected as described herein. FIG. 3A shows an overview (map) of one of the patient's jaws that was scanned by an intraoral scanner.

FIGS. 3B and 3C shows representative near-infrared (near-IR) and visible light channels, respectively, for one image from the set of scanned images. FIG. 3D shows the near-IR image of FIG. 3B indicating two caries centers identified as described herein. FIGS. 3E and 3F show x-ray images of the same region of the teeth shown in FIGS. 3B-3D; in FIG. 3F the caries are marked.

FIGS. 4A-4F illustrate an example of caries detected as described herein. FIG. 4A shows an overview (map) of one of the patient's jaws that was scanned by an intraoral scanner.

FIGS. 4B and 4C shows representative near-infrared (near-IR) and visible light channels, respectively, for one image from the set of scanned images. FIG. 4D shows the near-IR image of FIG. 4B indicating a caries center identified as described herein. FIGS. 4E and 4F show x-ray images of the teeth.

FIGS. 5A-5F illustrate an example of caries detected as described herein. FIG. 5A shows an overview (map) of one of the patient's jaws that was scanned by an intraoral scanner.

FIGS. 5B and 5C shows representative near-infrared (near-IR) and visible light channels, respectively, for one image from the set of scanned images. FIG. 5D shows the near-IR image of FIG. 5B indicating a caries center identified as described herein. FIGS. 5E and 5F show x-ray images of the teeth.

FIGS. 6A-6F illustrate an example of caries detected as described herein. FIG. 6A shows an overview (map) of one of the patient's jaws that was scanned by an intraoral scanner. FIGS. 6B and 6C shows representative near-infrared (near-IR) and visible light channels, respectively, for one image from the set of scanned images. FIG. 6D shows the near-IR image of FIG. 6B indicating a pair of caries center identified as described herein. FIGS. 6E and 6F show x-ray images of the teeth.

FIG. 7 illustrates an example of a user interface for detecting and/or displaying detected caries.

FIGS. 8A-8C illustrate an example of a user interface for detecting and/or displaying detected caries.

FIG. 9 illustrates one example of a method of determining which images from an intraoral scan are most likely to include caries, if caries are present in the teeth. This method may be performed prior to identifying caries within the scan.

FIGS. 10A-10D illustrate one example steps that may be part of a method of determining images from an intraoral scan that are most likely to include caries.

FIG. 11 shows an example of one step that may be included as part of a method of determining images from an intraoral scan.

FIG. 12 schematically illustrates an example of features that may determine from images of an intraoral scan for scoring the images as described herein.

### DETAILED DESCRIPTION

Described herein are methods and apparatuses (e.g., systems, devices, etc. including software, hardware and/or firmware) for identifying caries from scans of a patient's teeth. These methods and apparatuses may automatically or semi-automatically detect caries. These methods and apparatus may be used with an intraoral scanner, and in some cases may be included as part of the intraoral scanner. In some cases, these methods and apparatuses may receive scan information but are not necessary part of (or integrated with) an intraoral scanner.

Also described herein are methods and apparatuses for determining which images may best show caries from an intraoral scan, should caries be present by scoring the images of a scan (e.g., the near-IR images and/or visible light images) and identifying those that are most likely to have caries, without necessarily identifying caries in the images. The images most likely to have carries may be presented to a user (e.g., displayed, saved, transmitted) and/or may be used as an input into any of these methods for identifying caries.

In general, these methods and apparatuses may determine one or more features associated with a caries, such as a caries outline, caries center, etc. This feature (or features) may be identified from a plurality of different images from scans of the patient's teeth, e.g., using a pattern matching agent, such as a trained machine learning agent (“trained pattern matching agent”), which may indicate possible caries, also referred to herein as putative caries. These methods and apparatuses may determine a likelihood that a particular putative caries corresponds to an actual caries by using one or more additional techniques, including projecting a representative feature of the possible caries (e.g., caries centers) onto a 3D model of the teeth, and based on an analysis of a minimum number of overlapping scans of the patient's teeth showing the representative feature from different camera angles. Thus, the methods and apparatuses described herein may dramatically eliminate false positives and false negatives.

Also described herein are methods and apparatuses for outputting identified (e.g., automatically identified) caries in a manner that simplifies the analysis for dental professionals, and allows highly accurate tracking of caries across time (e.g., longitudinal analysis).

### Identification of Caries Center

In general, the methods and apparatuses described herein determine or detect dental caries, or a feature of dental caries (such as caries boundary, caries center, etc.). These methods and apparatus may be automatic or semi-automatic. Prior attempts to automate the detection of dental caries has proven difficult; prior machine learning methods for detecting caries typically do not work well, and often result in numerous false positives. Caries may be difficult visualize when examining a limited number of images, as the complex shapes of the teeth, as well as the orientation of the teeth may make imaging difficult, particularly within interproximal regions. The methods and apparatuses described herein may use multiple images, taken from different region of the teeth having overlapping fields of view, at a plurality of different imaging modalities (e.g., near-IR and multiple visible light wavelengths) and may use a trained pattern-matching (e.g., machine learning) agent that may identify one or more caries features (e.g., a caries center) of each of a plurality of caries from these multiple images having overlapping fields of view. The use of caries centers, and in particular the translation of the 2D images into a virtual 3D model of the patent's dentition and the use of projections of putative caries center points onto the virtual 3D model, including other techniques as described herein, may result in a statistically significant improvement in the quality and reliability of automatic caries detection as well as the speed of caries detection. In some examples described herein, caries centers may be referred to as caries center points. It should be understood that the caries centers are not necessarily limited to a single point but may refer to a region of any appropriate size. For example, a caries center (caries center point) may refer to a single pixel or (in 3D) voxel, or a group of pixels/voxels (e.g., 2, 3, 4, 5, 6, 7, 8, 9, 10, etc.). In addition, caries centers may refer to a geometric center of the caries region based on an identified caries boundary, and/or may refer to a center of the most severely affected region of the tooth (e.g., the center of a region of highest tooth decay), etc.

A general method for detecting caries may include taking or receiving a plurality of two-dimensional (2D) scans of a patient's teeth using an intraoral scanner. These scans may be used to generate a virtual three-dimensional (3D) model of the patient's dentition. In some examples, the 3D model may be concurrently constructed as the 2D scans are taken. Each scan may have a plurality of channels reflecting different wavelengths taken of the same (or approximately the same) region of the tooth, such as RGB (e.g., color), near-IR, fluorescent, viewfinder, etc. A viewfinder scan may refer to a visible light frequency that may be used for 3D surface topology (e.g., using structure light). Thus, for each 2D image scan there may be plurality of corresponding channels. Optionally, an image having a plurality of channels may be referred to as an image set, in which the set includes the plurality of channels. Each channel may itself be an image, such as a near-IR image, red light image, white-light image, etc. In some cases, the white-light image is a white-light image including red, green and blue channels.

Receiving the plurality of 2D images may include receiving a minimum of x images, where each image corresponds to an image set including a plurality of channels, such as NIR and RGB images (e.g., red, green, blue, separately, or in some examples collectively), etc. The images may include a plurality of images showing overlapping views of the same region of the dentition. Any appropriate number of images including the same overlapping region(s) may be used, including, e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 25 or more, 30 or more, 35 or more 40 or more 45 or more 50 or more, 55 or more, 60 or more, 70 or more, 80 or more, 90 or more, 100 or more, 110 or more, 120 or more, 130 or more, 140 or more, 150 or more, 160 or more, 17 or more, 180 or more, 190 or more, 200 or more, 250 or more, etc. Thus, in practice thousands, hundreds of thousands, etc., of images may be processed for caries detection as described.

In some examples, the plurality of images are collected in real time with the automatic detection of caries, or the automatic detection of caries may be performed after scanning is complete. Thus, in some examples, an intraoral scanner is configured to perform the automatic detection. For example, an intraoral scanner may include one or more modules (as described herein) for performing the automatic detection of caries and related processing techniques. Alternatively, or additionally, previously taken scans may be accessed (or may be received from a third party) by the apparatus for automatic detection of caries as described herein.

All or a subset of the plurality of scans may be processed to identify a caries center point (referred to for convenience as a caries center). The caries center point may refer a representative point on the surface of a tooth that is approximately the center of the caries on the tooth. In some cases, caries may have an irregular shape, thus the center point may be different between different images of the same region (and therefore same caries) of a tooth. The caries center point may be determined for each 2D image, and/or for each channel corresponding to each image set. As described below, the plurality of initially determined caries center points may be beneficially combined, e.g., aggregated, so that caries center points from different images and/or different channels showing the same regions of the teeth, and therefore potentially the same caries, may be combined in a manner that both confirms and refines the caries. This process, described in detail below, may greatly increase the accuracy and characterization of automatically identified caries.

The plurality of detected caries center points may the combined (e.g., aggregated) so that caries center points likely to be part of the same caries on the teeth identified in different images and/or different channels may be combined together. In some cases, this may include projecting the caries center points from all or a subset of the plurality of scans onto the 3D model of the teeth corresponding to the scans. In some cases, the 3D model may be segmented. In examples in which the plurality of caries center points are projected onto the digital 3D model of the teeth before aggregating the carries centers, the method or apparatus may determine which carries center points correspond to the same caries. For example, caries center points may be aggregated when they are within a predetermined distance of each other along the tooth surface, and within the same tooth segmentation boundary.

The apparatuses and method may then generate an indicator of one or more caries (e.g., an output) based on the caries center points that indicates the location of the one or more caries. In some cases, the indicator (e.g., output) may include a caries data structure. In some examples, the indicator (e.g., output) including a modified version of the 3D model including the consolidated caries center points and/or any of the associated caries data structure data. In some cases, the output is a report (e.g., a textual report) describing the one or more caries, including one or more of the location of the caries, the extent of the caries, the severity of the caries, and/or a confidence score for the caries. This report may be actionable by the user (e.g., dental professional) that may use the report to treat the patient's teeth based on the report. Treatment may include removing (e.g., filling) the caries, tracking/monitoring the caries, or otherwise treating the one or more caries.

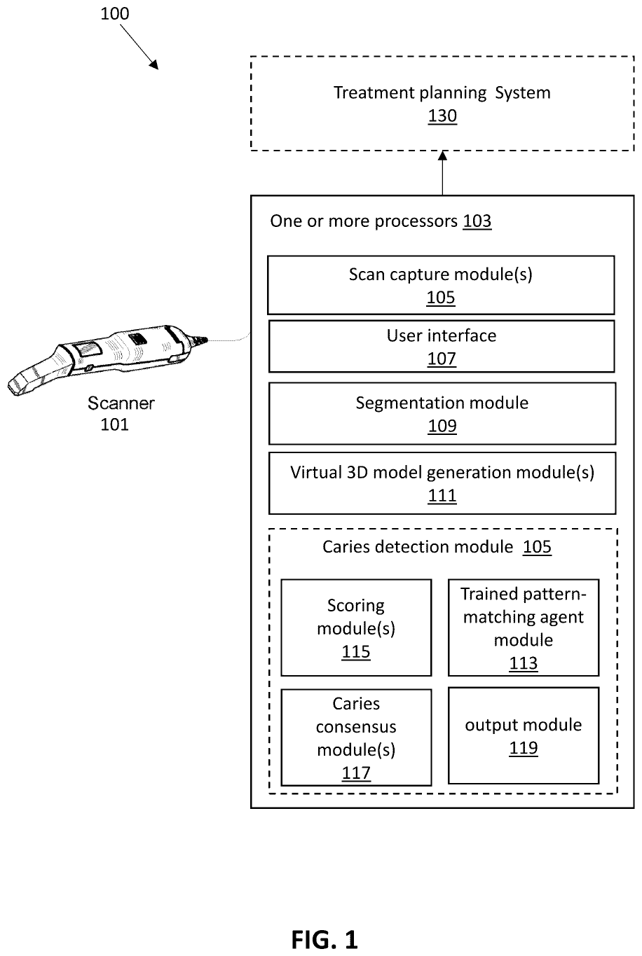

FIG. 1 schematically illustrates one example of an apparatus 100 for detecting caries. In this example the apparatus may include and/or may be used with an intraoral scanner. This is optional; in some examples, the apparatus may be used as a stand-alone system or device (and in some examples, software) that receives scans from the patient's teeth. Such examples may be similar the example shown in FIG. 1, but may not need the intraoral-scanning specific components, such as the scanner 101, scan capture module(s) 105, etc.

In the example shown in FIG. 1, the apparatus for detecting caries may include a caries detection module 105 that is functionally (and in some cases physically) connected to the one or more processors 103 that may be part of the intraoral scanner 101. The caries detection module may detect one or more caries from the teeth, and may provide an output for display, transmission, storage, etc. The output may be graphic, e.g., an annotate virtual 3D model of the teeth including caries or caries feature (e.g., caries centers, caries boundaries, etc.). Alternatively, or additionally the output may be textural, e.g., a written description of the caries location (tooth number and/or region of a tooth, coordinates of the caries, etc.), extent of the caries (e.g., size, volume, etc.), severity of the caries (e.g., severity score or rating), confidence score for the identified caries, etc. The output may summate caries data from across a number of scans of the teeth.

Any of these apparatuses may include a user interface 107 for interacting with the caries detection apparatus, and/or for displaying or otherwise outputting the detected caries. These apparatuses may receive pre-segmented images and/or 3D models of the teeth, including the scan images and/or 3D models. In some cases, these apparatuses may include one or more modules 109 for segmenting the images and/or the 3D model of the teeth. Alternatively, or additionally, the apparatus may generate a 3D model from the scan data using one or more virtual 3D model generation modules 111.

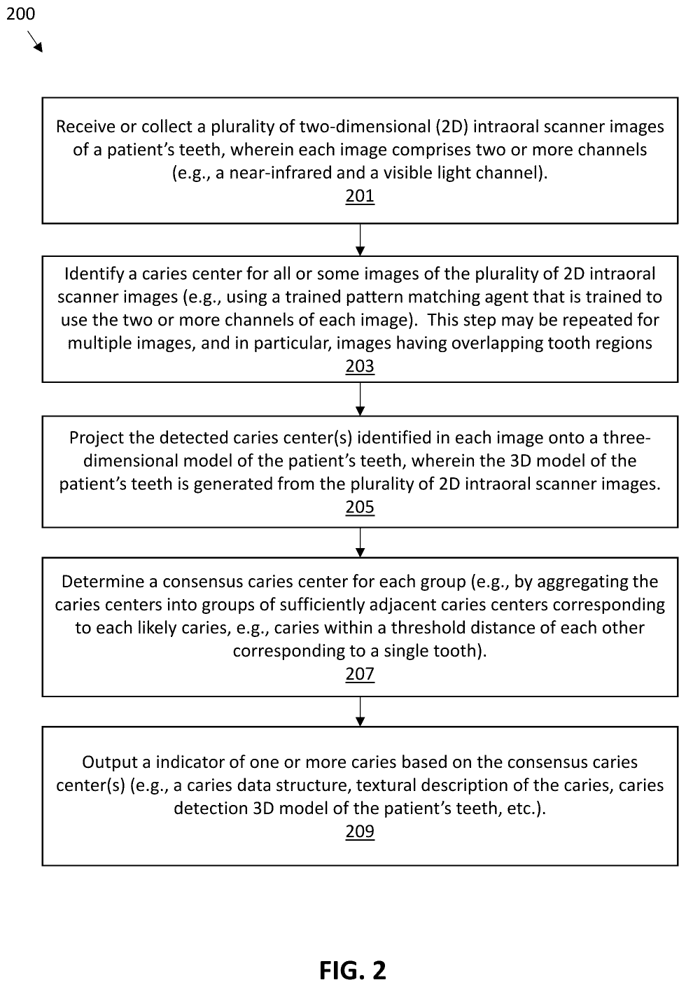

The caries detection module(s) 105 may include modules that may perform any of the methods described herein, as described in greater detail below, including in FIG. 2. For example, in FIG. 1 the caries detection module 105 may include control logic for coordinating automatic detection of caries using scanned images having two or more scanning channels (e.g., light wavelengths, such as near-IR, red, green, blue, infrared, etc.). The detection module may be configured to operate in real time (as images are collected) and/or after images have been scanned/collected. The detection module may also coordinate input and activity of the associated modules, including one or more trained pattern matching (e.g., trained machine learning) agent modules 113. The trained pattern matching module 113 may perform any or all of the functions of the trained pattern matching agents described herein. The trained pattern matching module may receive as input one or more images in which the image(s) has multiple channels, and may output caries information (e.g., caries center location(s), and in some case severity, likelihood, extent, etc.). The caries detection module(s) 105 may also include one more scoring modules 115 such as a severity scoring module and/or a confidence (or likelihood) scoring module, etc. Any of these caries detection modules may also include one or more caries consensus (or voting) modules 117. The caries consensus module may combine potential caries identified from different images having overlapping regions (e.g., overlapping regions describing the same possible caries from one or more of the images). The caries consensus modules may identify subsets of the scan images (e.g., of the images provided to the trained pattern matching module 113). In some cases, the caries consensus modules may weight or apply weights to these images during the combining steps.

The caries consensus modules may also project the characteristic of the caries (e.g., caries centers) from all or some of the scan image analyzed by the trained pattern matching agent onto the virtual 3D model of the teeth. In some cases, the caries consensus modules may include or may reference a caries projection modules to perform this.

In general, the caries detection module 105 may also prepare output including the detected caries information. The output may be configured to provide textural and/or graphic output. In some examples, the output module 119 may be configured to output to a patient file (e.g., medical and/or dental record) and/or medical/dental software or databases.

Any of the systems described herein may be used with or for treatment planning, including planning a dental and/or orthodontic treatment. For example, these apparatuses may include or connect to a treatment planning system 130 to assist a dental professional in treating a patient.

FIG. 2 illustrates one example of a method 200 for detecting caries. In this example, the method includes receiving or collecting a plurality of two-dimensional (2D) intraoral scanner images of a patient's teeth 201. Each 2D image may include two or more channels (e.g., a near-infrared and a visible light channel), as mentioned above. Caries center(s) may be identified for all or some images of the plurality of 2D intraoral scanner images 203 (e.g., in some examples, using a trained pattern matching agent that is trained to use the two or more channels of each image). This step may be repeated for multiple images, and in particular, images having overlapping tooth regions (e.g., for 2 or more images, for 3 or more regions for 4 or more regions, for 5 or more images, for 6 or more images, for 7 or more images, for 8 or more images, for 9 or more images, for 10 or more images, for 11 or more images, for 12 or more images, for 13 or more images, for 14 or more images, for 15 or more images, for 16 or more images, for 17 or more images, for 18 or more images, for 19 or more images, for 20 or more images, for 30 or more images, for 40 or more images, for 50 or more images, for 100 or more images, for 150 or more images, for 200 or more images, etc.). A minimum number of images having overlapping tooth regions may be used. These overlapping images may be taken from multiple different angles, relative to the tooth. In some examples, the minimum number of images is 2 or more, 3 or more, 4 or more, 5 or more, 6 or more, 7 or more, 8 or more, 9 or more, 10 or more, 11 or more, 12 or more, 13 or more, 14 or more, 15 or more, 20 or more, 30 or more 40 or more, 50 or more, 100 or more, 150 or more, 200 or more, etc.

Any of these methods may also include projecting the detected caries center(s) identified in each image onto a three-dimensional model of the patient's teeth 205, wherein the 3D model of the patient's teeth is generated from the plurality of 2D intraoral scanner images. The 3D model may be segmented to identify individual teeth. In some cases, the individual teeth may be numbered or otherwise identified. For example, the method may include projecting the detected caries center(s) onto a segmented 3D model. The projection may include overlapping or concurrent tooth centers. Projecting may be done virtually and/or computationally, and does not require generating an image or model showing the multiple, possibly overlapping caries centers (e.g., from each image and image channel, such as near-IR, red, green, blue, etc.) drawn on the 3D model, although such a model may be generated.

Any of these methods may also include determining a consensus caries center for each group of caries centers 207 corresponding to overlapping regions on the teeth. For example, consensus caries centers may be identified by aggregating the caries centers into groups of in which subsets of the plurality of caries centers that are within a minimum threshold distance from each other along a surface of the virtual 3D model of the patient's dentition while still being within the boundary of a particular tooth, following segmentation into the individual teeth. The minimum threshold distance may refer to a range sufficiently adjacent caries centers corresponding to each likely caries, e.g., caries within a threshold distance of each on particular identified tooth. Once identified the one or more consensus caries centers may be output 209, e.g., as a caries data structure, textural description of the caries, caries detection 3D model of the patient's teeth, etc.

As mentioned, any of the methods or portions of these methods may be performed by one or more modules including executable instructions. Modules may also be referred to herein as engines. A module may be implemented as an engine, as part of an engine or through multiple engines. As used herein, an engine includes one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors, or a first engine and a second engine can share one or more processors with one another or other engines. Depending upon implementation-specific or other considerations, an engine can be centralized, or its functionality distributed. An engine can include hardware, firmware, or software embodied in a computer-readable medium for execution by the processor. The processor transforms data into new data using implemented data structures and methods, such as is described with reference to the figures herein.

The modules described herein, and/or the engines, through which the systems and devices described herein can be implemented, can be cloud-based or local. As used herein, a cloud-based module or engine may run applications and/or functionalities using a cloud-based computing system. All or portions of the applications and/or functionalities can be distributed across multiple computing devices, and need not be restricted to only one computing device. In some embodiments, the cloud-based modules or engines can execute functionalities and/or modules that end users access through a web browser or container application without having the functionalities and/or modules installed locally on the end-users' computing devices.

As used herein, datastores are intended to include repositories having any applicable organization of data, including tables, comma-separated values (CSV) files, traditional databases (e.g., SQL), or other applicable known or convenient organizational formats. Data stores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Data stores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores described herein can be cloud-based datastores. A cloud-based datastore is a datastore that is compatible with cloud-based computing systems and engines.

The automated agents described herein may implement one or more procedures based on 3D virtual representations of teeth taken from subjects. A “tooth type,” as used herein, may refer to a specific tooth in the mouth of a human being. A tooth type may include any specific tooth identified according to an “anatomical tooth identifier,” which as used herein, may refer to any identifier used to anatomically identify the tooth type. Examples of anatomical tooth identifiers include identifiers of a universal or other tooth numbering system, character identifiers, image(s), etc. A “virtual 3D” model or representation may refer to a 3D rendering of a tooth or teeth. Examples of virtual 3D models include animated 3D renderings, composite 3D renderings assembled from 2D images, etc. A 3D virtual representation may have one or more “virtual surface contours,” or contours that define surfaces of the tooth in a virtual 3D space.

In various implementations, an automated tooth modeling engine(s) may implement one or more automated agents configured to describe 3D virtual representations of teeth with mathematical 3D descriptors that use spatial parameters. A mathematical 3D descriptor, as used herein, may refer to a mathematical function that represents virtual surface contours and/or other portions of 3D virtual representations of teeth according to spatial parameters. Examples of mathematical 3D descriptors include Elliptical Fourier Descriptors (EFDs), spherical harmonic functions that use voxelized spheres, and spherical harmonic functions that use non-voxelized spheres. A spatial parameter may refer to a parameter that relates to a spatial element. Examples of spatial parameters include coordinates, e.g., locational coordinates, identified along orthogonal systems, such as three translational planes, 3D polar coordinates, etc. As noted herein, mathematical 3D descriptors may parametrically represent a 3D virtual representation, or represent that 3D virtual representation according to one or more parameters, such as spatial parameters.

In various implementations, mathematical 3D descriptors may form a “mathematical 3D descriptor space,” or a datastore of mathematical 3D descriptors with descriptor locations assigned for each mathematical 3D descriptor space. “Descriptor locations,” as used herein, may refer to unique coordinates in the mathematical 3D descriptor space where each mathematical 3D descriptor reside. In various implementations, descriptor locations may be used to define “descriptor distances,” or differences in distances between descriptor locations of mathematical 3D descriptors in a mathematical 3D descriptor space. Detecting Caries

As mentioned above, any of these methods may include detecting caries using a trained pattern matching agent. The pattern matching agent may be trained on a dataset of intraoral scan images having two or more channels (e.g., four or more channels, such as R, G, B and near-IR, etc.). In general, the trained pattern matching agent may be trained to identify and output a caries center (e.g., caries center point) from the images.

In any of these examples, caries, or caries centers, may be detected as a point or region. The point or region may correspond to a point or region on a virtual 3D model of the teeth, which may be a 3D mesh model, which may include vertices, edges, etc.

The trained pattern matching agent may be an artificial intelligence agent, including a machine learning agent. The machine learning agent may be a deep learning agent. In some examples, the trained pattern matching agent may be trained neural network. Any appropriate type of neural network may be used, including generative neural networks. The neural network may be one or more of: perceptron, feed forward neural network, multilayer perceptron, convolutional neural network, radial basis functional neural network, recurrent neural network, long short-term memory (LSTM), sequence to sequence model, modular neural network, etc.

In some examples, a trained pattern matching agent may be trained using a training data set of a plurality of intraoral scans that have been reviewed and labeled to indicate caries and/or caries centers. In some cases, regions that are not caries have also been labeled. Each of the plurality of scans may include multiple images (having multiple channels, e.g., near IR, white light, etc.) and all or some of the images showing overlapping regions may be labeled. Images may include multiple labeled caries.

For example, a trained pattern matching agent may be configured to receive at least 2 or more (e.g., 3, 4, 5, etc.) input channels for each image, such as a near-infrared (Near-IR), one each of a red, green and blue channel, a florescent channel, etc. The trained pattern matching agent may then use the input to identify the caries centers (as a point or small region) corresponding to regions of potential caries in each of a plurality of images. This process may be repeated for a plurality of different images of the same (e.g., overlapping) regions. For example, the process may be repeated for tens, hundreds, thousands, tens of thousands, etc. of images having at least slightly overlapping fields of view as the scanner is moved over the patient's dentition. The separate images may be reviewed by the trained pattern matching agent independently. In some examples, separate, but slightly overlapping images (e.g., taken within a predetermined time and/or having similar camera positions within a proximity threshold) may be used by the trained pattern matching agent.

In some examples, the trained pattern matching agent may configured as a competitive neural network. For example, the trained pattern matching agent may be trained on both a positive neural network (indicating the presence of a caries and/or caries center) in an image) and a negative neural network, e.g., indicating regions that are not caries. This configuration may reduce the number of false positives. The apparatus may indicate the detected caries centers as potential or putative caries, and may provide a confidence score for the identified caries center. In some cases, the position of the caries center and/or the larger caries region may be stored (as well as any confidence score, etc.) and/or applied to a 3D model of the surface of the teeth (e.g., constructed from at least the images being analyzed). The positions of the caries/caries centers may be derived directly from the camera position, which may be included with the image data corresponding to the images. For example, each image from the intraoral scanner used to collect the images may have a camera position associated with it that may be accessed by the apparatus.

To improve the accuracy of any of the trained networks described herein, the network may be trained by negative labeling. For example, an existing AI network may be trained on second datasets the results given to labelers to mark where the algorithm is wrong (e.g., “negative labeling”); this data may then be feed back into the algorithm training to get an improved network with higher accuracy.

For example, a trained pattern matching agent may be initially trained as described herein on a labeled dataset and labelers may review the results. Labelers may mark only images where the agent is clearly wrong, e.g., by putting a mark of “nothing” where the trained agent was wrong, and may give extra attention to the image and put marks of any visible caries present (regardless if detected by the agent or not).

Alternatively, any of these methods and apparatuses may use automatic negative labeling by using the trained pattern matching agent on a new dataset, and projecting any detections (detected caries) from the 2D images to the 3D model, then projecting back from the 3D model to all images that overlap with this same region of the 3D model. In this case, wherever detection is done and result in a sufficiently small number of “positive” images that include overlap with the same region from the virtual 3D model, the detection may be tagged as “nothing” (e.g., false detection). For example, a trained pattern matching agent may detect a single image out of 4 or more images (e.g., 2 images out of 10 or more images) as including a potential caries image. The overlapping image may be marked as not including a caries.

Alternatively, these methods and apparatuses may use a network that focuses on the area of the detection but gets input from multiple images, as described herein. The data can be given by the images via projecting on the 3D surface and deprotecting on the image plane. The network may then determine and store the probability that the finding is correct (e.g., as a likelihood score or confidence score/level).

In any of these examples the trained pattern matching agent may be re-trained using the dataset enriched with the negative labeled images. In some examples, images labeled as not including caries (“nothing”) may be given negative weights. Images having caries labels (verified caries) may be given higher weights than in original images used to train the network. These techniques may reduce or eliminate errors that may otherwise arise from gingiva inflammation, spots and marks on teeth, tooth wear, and/or anomaly detection. For such indications, the initial marking may result in inconsistent labeling so that the system may benefit from a second round labeling.

As described in greater detail below, the caries and/or caries centers (and any corresponding data) across different images may be combined/aggregated to identify a single caries center for each caries or caries region identified by the trained pattern matching agent. This may generally combine the output of the trained pattern matching agent for a plurality of different images of the intraoral scan data examined. Each image may identify a portion of one or more caries, a single caries, multiple caries or no caries. In some examples, the apparatus may use the virtual 3D model of the teeth to aggregate the identified caries and/or caries centers. For example, these methods and apparatuses may use one or more projections to get a final caries/caries center and thus generate a single 3D detection flag corresponding to the caries center, for each caries. When aggregating regions of the teeth including caries, if the trained pattern matching agent determines that the likelihood of a caries is above a threshold, the trained pattern matching agent may indicate that the caries is present and provide an estimate of the carries center.

In some examples, these apparatuses may use the virtual 3D model as another input. The 3D model may be segmented prior to being used as an input and/or may be used unsegmented. In some examples, the 2D images provided as input may be segmented. Segmentation may be useful to differentiate between caries on different teeth, even when the teeth are close together. Alternatively segmented images and/or the 3D model of the teeth may be used after the trained pattern matching agent has processed the images.

In general, these methods and apparatuses may be used in real time, e.g., while scanning the teeth or shortly after scanning (e.g., during the same session).

As mentioned, detection of caries from the 2D intraoral scanner images of the patient's teeth may generally include scoring the 2D images with a confidence score that indicates a value of how likely that the detected putative caries actually corresponds to a caries. This confidence score may be generated as part of the trained pattern matching agent. In some cases, the confidence score may be generated using a confidence scoring module that generates an estimate for the confidence score based on one or more of: the morphology of the caries region detected (e.g., shape, size, etc.), the imaging values for the detected putative caries regions, how similar the putative caries region is to one or more of the training images, etc. In some examples, the carries data structure and/or the 3D model including the indicator of one or more caries may include a reference to one or more images from the plurality of 2D intraoral scanner images that best illustrates the caries, e.g., that has a highest confidence score. For example, any of these methods and apparatuses may associate a “most relevant image” from the 2D intraoral scanner images for each detected caries; this most relevant image (or example image) may include the two or more channels associated with the image (e.g., RGB and NIR channels). Thus, any of the user interfaces described herein may be configured to allow a user to select a putative caries and may display the one or more most relevant images and/or the corresponding channels associated with the most relevant image(s).

Thus, in general, the trained pattern matching agent may be configured to detect one or more caries center from each image (which may include multiple regions) and may be configured to score each caries, e.g., each caries center, for example, to indicate one or more metrics such as severity, confidence, size, etc.

Inputs into the System/Method

In any of these methods and apparatuses (e.g., devices and systems, including software, hardware and/or firmware) the input for carries detection may be based on an intraoral scan of the patient's teeth. In particular, the input may be part of an intraoral scanner that includes a near-infrared (near-IR or NIR) scanning as well as visible light (e.g., red, green, blue, or RGB) imaging. In some cases, the scanner may also include one or more additional wavelengths, including fluorescent wavelength(s), and/or a viewfinder wavelength. For example, see U.S. Pat. No. 10,507,087, herein incorporated by reference its entirety. The intraoral scanner may concurrently or sequentially (in an alternating manner) scan one or more images at each of these wavelengths and may build the 3D model. The camera positions may be recorded during each scan. Scans of multiple different wavelengths may be combined or integrated into a single 2D image or a set of 2D channels referred to as a single image. Each image may cover approximately the same region of the oral cavity.

In some examples, additional scanning modalities may be included as well (not limited to near-IR, visible light, fluorescent, etc.). For example, these methods and apparatuses may be used with a second source or scanning modality, such as a dental X-ray, which may be taken, for example, within a short period of time (minutes, hours, days) of the intraoral scan of the specific patient. Thus, these various types of scan data may be combined for the dentist, providing an integrated (“all-in-one”) experience for practical and easy clinical decision-making when reviewing a patient's dentition. In any of these methods, the different modalities (e.g., different channels) may be adjusted or otherwise compensated as they are taken from slightly different camera positions as the intraoral scanner is moved.

In some cases, an additional modality may be used to identify the caries as described, e.g., may be provided to confirm the presence or absence of caries in a particular region of the teeth. Additional sources of data, such as X-ray data, may be used to set a confidence score for the identified caries. In some cases, the X-ray data may be used to set a sensitivity to finding a caries from one or more other sources of data (e.g., 2D images taken as part of an intraoral scanning system. The supplemented data (e.g., X-ray data) may also or alternately be used to inform the analysis of one or more tooth regions. For example, the methods and systems described herein may be configured to adjust their sensitivity to one or more regions (e.g., specific areas) based on an algorithm that uses X-ray data of the same tooth or teeth region; if the X-ray data does not indicate the likelihood of caries within a region, the method or apparatuses described herein may adjust the sensitivity for that specific area, to be sure that no caries will be missed.

Additional Inputs for network may include additional channels showing scan data. For example, although four channels are illustrated herein (see, e.g., Example 1, below), in some cases, additional or fewer channels may be used. For example, in some cases, instead of four channel, additional channels, such as the output of an MTD network or any calculation from such a network may be used. An MTD network returns, for every color image pixel, 7 channels with probabilities to be one of 7 categories. In this example 11 channels may be used as input to the trained pattern matching agent. One or more additional channels may be used for input, e.g., by producing a mask (zeros and ones) of whether the probability to be of rigid category is higher than the probability to be of non-rigid category. Any intermediate combination of these channels may be used.

Alternatively, or in addition, tooth metadata, such as tooth number, anterior/posterior, crown/original tooth/permanent or deciduous tooth, can be used also as input to the trained pattern matching agent.

In some examples, the pattern matching agent may be trained with several images as input. For example, a convolutional neural network (CNN) may be used on each image separately and then the results may be combined by using a fully connected network. The images may be projected to common planes and used as an input to the pattern matching agent. In some examples, a produce volumetric field may be used by using, for example, NERF and the nerf weights may be used as an input to the network.

Any of the methods and apparatuses described herein may use combinations of scan images (e.g., composite images) rather, particularly in instances where the individual images may have relatively small fields of view. Thus, any of the methods and apparatuses described herein may be used with images in which two or more images having adjacent and/or slightly overlapping fields of view may be combined to form a single image. Multiple channels may be used, as described above.

Alternatively, or in addition to the use of intraoral scan data (2D images) as described above, in some examples 2D images may be provided by remote scanning using 2D images taken of a patient's dentition using, e.g., a mobile phone or other imaging device that is not necessarily an intraoral scanner. In some cases, remote scanning (e.g., using a mobile phone) may be used in combination with a 3D model generated by intraoral scanner. In some cases, 2D images taken by a mobile phone using an attachment for imaging in both color images and near-infrared (NIR) images may be used. This may allow the patient to self-monitor and take images of these locations. This may further allow tracking by the patient and/or dental professional using the methods and apparatuses described herein. For example, images taken using this technique may be aligned or referenced relative to an existing 3D model.

### Consensus Confirmation of Caries to Eliminate False Positives

In general, the methods and apparatuses (systems) for performing them may confirm that caries identified by the trained pattern matching agent is legitimate, and actually corresponds to a potential caries. This may be accomplished by combining information from multiple images of the same region of the teeth, e.g., minimum of x sets of images, where the set of images include NIR and RGB images (x may be 10 or more, 15 or more 20 or more, 25 or more, 30 or more, 35 or more 40 or more 45 or more 50 or more, 55 or more, 60 or more, 70 or more, 80 or more, 90 or more, 100 or more, 110 or more, 120 or more, 130 or more, 140 or more, 150 or more, 160 or more, 17 or more, 180 or more, 190 or more, 200 or more, 250 or more, etc.).

In some cases, the trained pattern matching agent may provide a confidence indicator (e.g., a score indicating confidence, such as a percentage score in which the higher percentage indicates a higher confidence). The confidence score may be associated with each identified caries center point Alternatively or additionally, the trained pattern matching agent may provide a confidence score that caries are not present in a particular image or region(s) of an image.

The output of the trained pattern matching agent may be processed to confirm, for each tooth region and/or for each identified caries center points, that any putative identified caries center points actually corresponds to a caries. In some cases, this may include combining sufficiently proximate identified caries center points into a combined identified caries center point and/or generating a consensus confidence score for the combined identified caries center point.

When projecting the one or more caries centers (e.g., caries center points) onto the 3D model, the methods and apparatuses described herein may project the caries centers onto the 3D model by identifying the surface of the model (e.g., from the mesh, vertex, etc.) and may project a ray that extends through the camera (e.g., corresponding to one or more images including the carries center) to the specific image location of the carries center and may project the caries center onto 3D model surface for each carries center. As mentioned where two or more identified putative caries centers are identified are sufficiently close to each other in space, e.g., so that the boundary of the putative caries are overlapping, e.g., above a threshold amount, and/or the caries centers are within a threshold proximity) they may be identified as being part of the same caries region. The putative caries regions may then be combined, e.g., aggregated. The methods and apparatuses may use the segmented 3D model (or in some cases, 2D images the putative caries region(s)) to confirm that the putative caries region(s) and/or caries centers are on the same segmented tooth structure.

In some examples, the identified caries center point may be combined based on proximity, either absolute proximity or relative proximity. For example, the identified caries center point may be combined based on proximity as determined from their numeric coordinates. In some examples, the identified caries center point may be combined based on their relative proximity on a surface of the tooth. For example, identified caries center point may be combined using the 3D model of the teeth, by e.g., mapping the identified caries center point onto the 3D model and using the 3D surface to confirm that identified caries center point are sufficiently close to each other to combine. The putative caries center points may be positioned on the 3D surface of the 3D model. For each image taken, the camera position may be known, either using camera positions of the images (the scanner, either from intrinsic, e.g., position sensors) and/or extrinsic camera matrices allow the identified center of the caries for each 2D image to be positioned onto the 3D model. When positioning the identified caries center points, duplicates may be grouped, e.g., by requiring a minimum distance relative to each other. This minimum distance may be a minimum distance between sections of the camera vectors at the approximate position of the teeth (e.g., at a fixed distances from pinhole) for each identified caries center point. The position of the caries in space may be derived from the camera position. The position of the caries may be determined relative to the surface of the tooth in 2D or 3D.

In any of the methods and apparatuses described herein the method may use a plurality of images (e.g., a minimum number of images) in order to improve the statistical precision for the caries center point and/or for combing caries. The methods and apparatuses described herein may use multiple (e.g., 2 or more, 3 or more, 5 or more 10 or more, 15 or more 20 or more, 25 or more, 30 or more, 40 or more, 50 or more, 60 or more, 70 or more, 80 or more, 90 or more, 100 or more, 120 or more, 150 or more, 200 or more, etc.) images of the same general region of the teeth to identify and/or characterize a dental caries, and in particular, to identify the caries center point(s). A plurality or images covering the same general (or overlapping) regions including the dental carries may be used for identifying the caries (e.g., caries center point), including being reviewed by the pattern matching agent. The methods and apparatuses described herein may generally track the regions of the teeth and which sets of images (e.g., including multiple channels covering approximately the same region(s)) correspond to the same regions and may associate dental carries within approximately the same region. In some cases, the methods and apparatuses described herein may prioritize images with duplicated detections by the highest detection confidence provided by the detection algorithm. In some cases, the methods and apparatuses may provide a confidence score for the putative caries identified (e.g., putative of caries center point(s)); at least a part of the confidence score may account for the number of 2D image sets (referred to herein as images, for convenience). Thus, for regions having multiple images, taken from different positions, the confidence score may be higher. Other factors that may contribute to the confidence score may be set by the pattern matching agent (e.g., based on how robust the pattern matching is), and/or based on the location of the caries, the extent of the caries, etc.

In any of these examples, when mapping a group of caries center points identified from the 2D images to a single 3D digital model of the dentition, the methods and apparatuses described herein may identify caries center point from images with the highest confidence score from the group, or by applying a selection prosses in which only images having caries center point that have a confidence score above a threshold may be used.

As mentioned, caries center points that are near each other may be combined into a consensus caries center point based on an applied threshold for how near the caries center points are relative to each other. In some cases, the combined caries center points may combined when they are within a threshold distance from each other, e.g., along a surface of the 3D tooth model. The consensus caries center point may be at a geometric average of the combined caries center points, or it may be positioned at a weighted average based on the relative confidence score for the caries center points being combined. The confidence score for the combined or consensus caries center point may be determined based on the individual confidence scores for the caries center point(s) and/or based at least in part on the images including the region including the caries center point.

In some cases, the consensus caries center point may be determined from the plurality of caries center points identified using either or both the 3D model of the teeth that has been segmented into individual teeth (and in some cases including gingiva, etc.) and/or of the sets of 2D images that have been segmented to identify teeth and/or other oral components (gingiva, palate, etc.). In any of these methods and apparatuses, caries center points may be combined only when they are part of the same segmented regions, e.g., same tooth. In any of these examples the segmented 3D model and/or 2D images may be used before identifying the caries center points (e.g., using the pattern matching agent) or after identifying the caries center points. Segmentation may be performed in any appropriate manner, including using a separate trained pattern matching agent and/or using one or more algorithmic techniques.

Any appropriate segmentation technique may be used. For example, when projecting the caries center points onto the 3D model of the patient's teeth (which may be derived from the 2D scans used to identify caries center points) the 3D model and/or 2D images may be segmented, as mentioned above. The caries center points may be added to the 3D model after combining to form a combined/aggregate caries center point from sufficiently close caries center points, or they may be added before combining, and the projected caries center points on the 3D model may be used to help combine/aggregate them, as mentioned.

Thus, in some examples, one segmentation technique may use a trained pattern matching agent (e.g., a convolutional neural network such as an MTD network) which can separate teeth in 2D images. By projecting the direction of jaw to the image the methods or apparatuses may find an image in which part of the tooth on the image is mesial and which part is distal. Two neighboring caries from two different teeth of either sides may therefore not be aggregated together.

One segmentation method that may be used may include the use of a neural network which can separate teeth in images. By projecting the direction of jaw to the image these methods and apparatuses may find on the image which part of the tooth on the image is mesial and which part is distal. Two neighboring caries from two different teeth of side of tooth would therefore not be aggregated together, as mentioned above.

In some examples, the methods and apparatuses may segment the 3D mesh (provided by the intraoral scanner) using a technique that also provides tooth numbers. The tooth number can be further approximated to mesial and distal surfaces by getting the distance to the nearby teeth (e.g., accounting for missing teeth and last molar). By sampling each point on the images (e.g., using camera matrix), these methods and apparatuses can get the intersection with the mesh, e.g., by ray tracing, and get the image segmentation, so each point on the image corresponds to a specific surface (e.g., tooth number+mesial/distal). Using the image segmentation the method may separate detection, and perform non-max-suppression for every surface included in the image separately.

As mentioned, in cases in which caries are present on both a mesial region of tooth N, and distal region of tooth N+1, which are very close to each, detections may be mistakenly grouped into a single 3D detection. By using teeth segmentation info, these systems and apparatuses may identify that these detections should be separate and not grouped together, and show them as two different detections. Thus, using the teeth segmentation info, and with teeth numbering, these methods and apparatuses can get a textual clinical description of the caries (e.g. “Mesial of ADA 5”), as described in greater detail below. This information may allow the creation of an automatic report for the dental professional that may include information for each of the caries, including their description and optionally a snapshot (image or portion of image/channel) that best shows the caries, such as a portion of an image having the highest confidence level of detection. This data may be shared with a dental records systems, so a dental professional will have access to the detections along with patient information management.

In some examples, the segmentation info may be projected from 3D to the 2D images (or vice versa). The methods and apparatuses described herein can show to the doctor the tooth number, the tooth directions (buccal/distal, etc.) and tooth borders on the near-IR images, which may help the dental professional's orientation when looking and analyzing the near-IR images that can sometimes be confusing.

During operation, the apparatus may identify or determine 3D surface points from 2D image predictions. For example, using camera positions of the scanned/scanning images (provided by scanner, e.g., both intrinsic and extrinsic camera matrices) the apparatus can group duplicates by setting and/or requiring a minimal distance between sections of the camera vectors at an approximate position of the teeth (e.g., fixed distances from pinhole). In some examples, the apparatus may further require a minimal number of images including the detection (which may improve the precision statistics). For example, the apparatus may prioritize images with duplicated detections by the highest detection confidence provided by the detection algorithm.

In any of these methods and apparatuses, when mapping a group of 2D detections to a single 3D detection, the apparatus may identify an image with the highest confidence detection in the group.

Alternatively, as mentioned, the methods or apparatuses may segment the 3D mesh (e.g., the 3D digital model of the tooth). In some examples, these methods and apparatuses may include identifying a tooth number for each tooth. The tooth number can be further approximated for mesial and distal surfaces, e.g., by determining a distance to the nearby teeth, and/or may account for missing teeth and the last molar. By sampling each point on the images, e.g., using a camera matrix, these methods and apparatuses may determine an intersection with the mesh (e.g., ray tracing), and get the image segmentation, so each point on the image corresponds to a specific surface, including tooth number and mesial/distal. Using the image segmentation, these methods and apparatuses may then separate detection, and perform object detection (such as, but not limited to non-max-suppression) for every surface included in the image separately.

Thus, the use of segmentation as described herein may allow more accurate identification of caries even between closely positioned teeth. For example, in cases in which caries are present on both a mesial region of a first tooth (N) and a distal region of an adjacent tooth (N+1), when the teeth are very close to each, which may otherwise cause the caries detections to be grouped together to a single 3D detection, these methods and apparatuses may be able to separate the individual caries. The use of the segmentation may allow the caries detection to separate (rather than group together) even closely adjacent caries, so that they may be shown as two different caries detections.

In some examples, the methods and apparatuses may include allowing a user (e.g., technician, dental professional, etc.) to review the identified caries center points. For example, a user may be shown a representation of the caries center point(s) on the 3D model of the teeth and/or the 2D images. The user may be shown the labeled model/image either for confirmation (to allow the user to agree/disagree) or as output. In some cases, the user may confirm (e.g., choose, select, etc.) the specific caries center point on the 2D image or 3D model. The methods or apparatuses may show to the user a 2D image (or multiple channels of the image, such as an image pair including an RGB image and a near-IR image) in which the detection is best seen. In some examples, the representation of the 2D image(s) may be shown when moving a window (e.g., loop, etc.) over the 3D model to one or more 2D images that may be used to show more or less detail. In some examples, one or more 2D images having the highest confidence score may be shown a representative image of the caries center point.

These methods and apparatuses may be used with one or more additional data-driven user interfaces, and in particular one or more data-drive AI procedures for reviewing and analyzing intraoral scans.

### Voting

As mentioned, in general these methods and apparatuses may use a voting technique to confirm that a putative caries is a caries and/or to determine when to aggregating putative caries. These methods and apparatuses may process each of a plurality of 2D intraoral scan images and may identify each individual image as having no putative carries, one putative caries, or multiple putative caries. The methods described herein may generally therefore include a large number of images having overlapping regions of the teeth, covering the same putative caries from multiple (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 25 or more, 30 or more, 35 or more, 40 or more, 50 or more, 60 or more, 70 or more, 80 or more, 90 or more, 100 or more, 110 or more, 120 or more, 130 or more, 140 or more, 150 or more, etc.) different views (e.g., different camera angles). In practice typically less than all of these different views only a subset of these different views of the same region may be detected (e.g., by the trained pattern matching agent or otherwise) may indicate the presence of a caries. The methods and apparatuses described herein may review the presence and/or absence of detected caries from overlapping regions of the teeth taken from this plurality of different camera angles to provide a confidence score for the putative caries, and/or to determine if a caries is present, and if so, what the likely caries center is.

For example, if the trained pattern matching agent determines that only a small number or proportion of the overlapping 2D intraoral scanner images indicate a putative caries (e.g., caries region and/or caries center, but similar and/or overlapping views do not indicate putative caries (e.g., less than 10%, 15%, 20%, 25%, 30%, 40%, 45%, 50%, etc.), the apparatus and methods may indicate that a caries was not detected for this region (e.g., “no detection”). In some examples, the number or percentage of overlapping 2D intraoral scanner images for a particular region of the 3D model that indicates caries and/or does not indicate caries present may be used to set a confidence value for the region. Thus, in general, these methods may ‘vote’ based on the plurality of different regions to indicate that caries are or are not present, and/or the extent of the caries.

In any of these methods and apparatuses, the method may indicate a severity of the caries detected as part of a severity map. In general, a severity map may provide a location-based score (e.g., as a heat mapping) of the caries severity and/or an average severity score for the overall caries (within the boundary of the caries detected). A severity map may indicate which pixels (and/or voxels) corresponding to tooth image; the severity of the caries at each point (pixel, voxel, etc.) or sub-region may be determined and tracked, e.g., in a separate data structure and/or as part of the 3D model and/or as part of the caries data structure. The severity of a caries may be based on the images data for all or some of the channels. For example, the severity of a caries may be based on the intensity (or normalized intensity) of the near-IR image of the tooth at a particular region and/or the RGB image values (intensity, color, etc.). For example, near-IR images may identify caries and color images (e.g., RGB image or images) may be used to add accurate color data which may be used to determine tooth staining, gum inflammation, etc., which may be used to indicate severity of the associated caries. The data from these multiple imaging modalities (channels) may be incorporated into the severity mapping/score. Tracking the severity map may allow the methods and apparatuses described herein to track progression of caries over time. Although in general, caries do not typically reduce in size, caries may stop growing (e.g., arrested carries). Any of the methods described herein may be configured to track the change in caries over time. For example, the methods and apparatuses described herein may use time lapse to track the progression of caries over time; including comparison of the size, shape and/or severity of caries over time.

In some cases, the position of the camera when taking the 2D scan images may impact how likely it is to detect one or more caries from the image. Thus, in some cases, proximal caries (e.g., between the teeth) may be more difficult to detect from buccal or lingula views, and therefore occlusal views may be preferred, and may be weighted more by the detection module (e.g., the trained pattern matching agent). Thus, occlusal views may be given a somewhat greater confidence value for detected (or not detected) caries.

### Severity of Caries

As mentioned, the methods and apparatuses described herein may determine an indicator of the severity of the caries detected. The severity may be determined independently or jointly with a confidence score for each of the caries identified (e.g., by each individual center of caries and/or by a collective center of caries). In some cases, as mentioned above, the trained pattern-matching agent may be trained to identify the severity of the caries when identifying the caries and may therefore determine a putative severity score. The severity score may be combined with, separate from and/or informed by the confidence score. Alternatively, or additionally, a severity score may be based on a classification of the identified caries region as part of a separate manual, automatic or semi-automatic technique for determining the severity of the caries once identified from the 2D images and/or once mapped to a 3D digital model of the patient's teeth. In some cases, the severity of the caries may be determined using a separate classifier, such as a trained pattern-matching agent (or system or sub-system) that ranks or scores caries once the center of the caries has been identified (e.g., using the trained pattern matching agent for identifying centers of caries). As mentioned, this may include an estimate of the classification of the caries severity (e.g., E1/E2, etc.). Any appropriate scoring system may be used, including American Dental association (ADA) caries classification, such as the E0-E2, D1-D3 notation system, e.g., E0 (no lesion), E1 (lesion within the outer half of enamel), E2 (inner half of enamel), D1 (outer third of dentin), D2 (middle third of dentin), and D3 (inner third of dentin). Any other appropriate scoring system may be used.

As mentioned, the methods and apparatuses described herein may automatically find the size of the caries and their location. This may be done by several techniques, training of bounding boxes labeled data, grad-CAM, etc. In any of these methods and apparatuses the method may project the area of the caries from the specific figures to the 3D model getting severity map on the 3D surface or alternatively producing volumetric model of the caries. This may provide an estimate the classification of the caries severity, as discussed. (e.g., E1/E2, etc.)

Caries severity may be tracked longitudinally (e.g., over time).

### Extent of Caries

In addition to the center of the caries, the methods and apparatuses described herein may determine the extent (size, shape, position, etc.) of the caries identified by each center of caries. Thus, these methods may automatically determine the size of a caries and its location. This can be done by several techniques, training of bounding boxes labeled data, grad-CAM, etc. As mentioned, the size of the caries, confidence score, severity score, and/or center of the caries may be stored as part of a data structure that may reference and/or may be part of a 3D model of the patient's dentition. In some examples, these method or apparatuses may project the area of the caries from the specific 2D image(s) onto the 3D model, providing an extent and/or severity map on the 3D surface or alternatively producing a volumetric model of the caries.

### Caries Data Structure

Each caries may be stored as part of a caries data structure and/or a part of a 3D model of the teeth. The caries data structure may include an entry for each identified combined/aggregate caries, indexed by the identified caries center point. The caries data structure may be part of a database that may be separate from the 3D model of the patient's dentition or may be integrated with the 3D model of the patient's dentition. Within the caries data structure each caries center point (or, in some examples, an aggregate/combined center of caries) may be correlated with the related values, such as a confidence score specific to each caries center point, an indicator (e.g., identifying the points/voxels making up the putative caries associated with the caries center point, dimensions/parameter values specific to the caries associated with the caries center point, coordinates of the caries associated with the caries center point and/or of the caries center point itself, etc., and/or a surface area and/or volume estimate for the caries associated with the caries center point) of the extent of the caries on the tooth, an indicator of the severity of the caries, an indicator of a particular image or images that illustrate the caries, a marker or comment added by a user, a tooth number on which the caries is present, a location on the tooth or set(s) of teeth (e.g., interproximal, etc.), etc.

### Progress Tracking

In general, the methods and apparatuses described herein, including method and apparatuses for caries detection, may also or alternatively be used to detect changes in caries (both in 3D and in 2D images) over time to estimate the caries progress. This may be done by analyzing multiple different scans taken at different times.

### Automatic Reporting

Any of the methods described herein may include automatic reporting and in particular automatic textual reporting on caries. In general, the automatic reporting may or may not include the 3D virtual model of the patent's dentition onto which the potential caries (e.g., the caries center point(s)) are indicated. For example, in some variations, the output of the apparatuses and methods for automatically detecting carries may include, e.g., as part of a user interface, described in greater detail below, a virtual 3D model of the patient's dentition. A projection of the 3D model may be shown and manipulated by the user (e.g., via an input, such as a mouse, keyboard, touchscreen, button, etc.) which may optionally change the orientation of the 3D model of the upper and/or lower jaws, including the teeth, and in some cases, gingiva, palate, etc. In some examples, a movable window may be positioned over the 3D model and may be used to select a region for viewing, using one or more of the channels (e.g., color image, NIR image, viewfinder image, fluorescent image, etc.) corresponding to the region of the 3D model being shown. The image(s) shown may be actual images or may be synthetic images generated from the 3D model. In some examples, the 3D model may include annotations (on or adjacent to the model) indicating caries. The user may choose to see all of the caries (e.g., the aggregate caries), or one or more subsets of caries such as just a subset of caries having a confidence score above a threshold (which may be user-adjustable or preset) may be shown, and/or caries having a severity score above a threshold (which may be user-adjustable or preset), or of a particular class or type, may be shown. In some examples, the user may select to display carries having a particular extent may be shown. In some examples, the user may select one or more regions of the 3D model to display caries. Caries may be shown on the model by a marker (color, text, shape, etc.) and may be selectable to provide additional detail, e.g., when selecting and/or moving a cursor over, etc. The additional information may include any of the information from the data structure corresponding to a particular caries center point (e.g., confidence score, severity, etc.). The additional information may be continuously displayed. In some examples, the information about the caries may be shown as table, graph, chart, listing, etc. In some examples, the particular dental caries (e.g., aggregate or combined dental caries centers) may be selected to display one or more images or sets of images that illustrate the dental caries particularly well, which may also be part of the caries data structure. The one or more images may be shown alongside of the 3D model. The display of the 3D model may be marked to show just the center of the caries point and/or it may be marked to show an outline of the extent of the caries on the 3D model.

Alternatively, or in addition, any of these methods and apparatuses may be configured to generate a textual report of all or some of the caries. The user may select a subset of the caries to display, either manually (e.g., by interactively selecting specific dental carries from the aggregate/combined dental caries center's shown on the 3D model), automatically (e.g., providing a listing of all or a subset of dental caries based on one or more property of each identified aggregate/combined dental caries center point, such as confidence score, extent, severity score, location/tooth number(s), etc.), or semi-automatically, in which the user may specify one or more properties (location/tooth number(s), severity, confidence score, extent, etc.) to generate a textural listing including an ADA or other generally understood rating scale (e.g., E0-E2, D1-D3 notation system). A clinical textural module may synthesize the data associated with each (or a subset) of the aggregate/combined caries center points into clinically appropriate language. Thus, the methods and apparatuses may use any of the information from the caries data structure including, the tooth number, severity, etc. to generate textual descriptors from the caries data structure that are clinical descriptions of the caries. These methods and apparatuses, including the clinical textural module, may create an automatic report for the dental professional that may include for each of the caries identified (or a selected subset of them) a textural description of the caries. In some examples, the report may also include one or more images (e.g., a projection of the 3D model, and/or a 2D image selected from scan data, and/or a synthetic image generated from the scan data or 3D model) showing (highlighting, coloring, etc.) the caries. As mentioned, the report may include an image that best shows the caries (such as an image having the highest confidence level of detection from the images including the particular caries center point).

The report may be output including output for transmission (e.g., to a remote server, to a patient medical/health/dental record, etc.) for display, for printout, etc. The report may be particularly helpful for adding to a specific patient's chart, and/or for later review.

In general, the data output from the methods and apparatuses for automatically detecting caries may include the caries database and/or 3D model and/or report and may be specific to the patient at the date/time of the scan analyzed (after or during the scan). In some examples, the data may be shared with a dental medical records system that may be integrated with the intraoral scanner, so that a dental professional (e.g., dentist, doctor, etc.) may have access to the caries detected when reviewing patient information, which may improve patient information management.

Thus, in general, any of these methods and apparatuses may provide clinical context to a user base on the automatic detection of caries as described, including creating a report. The report may summarize (in text) the scan information, which may save the clinician a significant amount of time. For example, a report may indicate in text where one or more caries is located (which tooth, by name/number) and/or the likely severity and/or extent of the caries. This report may also be provided (or a redacted specific version thereof) to a patient insurance provider. In practice the textural report may also allow the user (e.g., doctor) to look just at the summary, without having to manipulate the 3D model and/or images of from the scan. This database textual summary provided to the user (dentist, etc.) may include textural a summary report rather than just an image, such as (for each caries center point, e.g., each combined caries center point, or a subsection of the caries center points) a textural description identifying the issue (e.g., cavity), assign a clinical severity, identify the tooth (e.g., by tooth number and/or name) corresponding to each caries.

This output report may be referred to as a scan report, textural report, caries report (e.g., automatic scan report, automatic caries report, etc.) and in some examples, may be part of a diagnostic test or tests, and any identified results may be shown to a dental professional immediately or stored (e.g., in a medical record) for later review. In any of these examples the resulting textural report may be included as part of a user interface that may allow a user (e.g., dental professional) to modify or enhance the report, including adding additional textural comments. The output report may be generated automatically or may be selected for automatic generation by a user. For example, the apparatus or method may be configured to automatically generate a textural report upon completion of the scan, or following triggering by a user selecting to automatically identified caries.

The indicator of one or more caries may include a tooth number, and/or location, a location of the caries on the tooth, a size and/or shape of the caries, a location of the caries center, etc.

### User Interface

As mentioned above, any of these methods and apparatuses may include a user interface for displaying one or more images of the patient's dentition (e.g., scan images, synthetic images derived from scan image, 3D digital model (or projections of the 3D digital model) based on the scan images, etc. In general, these user interface may allow the user to control (run, modify/select options, etc.) for automatic detection of caries as described herein. In some examples, the user interface may be optimized for caries detection and/or the display of caries detected. In some examples, the software may enable a user (e.g., dentist, technician, or other medical professional) to view the entire dentition, both arches side by side, with all detections visible on the 3D models. The user may choose one or more detected caries and focus on them.

In some examples, the user interface may present detected caries to the user in a predetermined or selectable order, to ensure no detection is missed. The user may have a detection sensitivity control, e.g., to select to display only those caries having a sufficiently high confidence score (or within a selected range of confidence scores). The user may define their preference for a default sensitivity when opening a case for review, or a default sensitivity may be used. In some examples, the user interface may be configured to enable a user to select (e.g., tag) one or more caries with one or more comments or fields, such as selecting those caries that may be treated, or planned for follow-up, or for indicating/removing them from the identified caries (e.g., incorrect identification). For example, when a caries is marked follow-up, on the next visit, the system may prompt a display or presentation of the same region of the tooth/teeth including the caries so that user can check that region again. The tags may be included in the caries data structure.

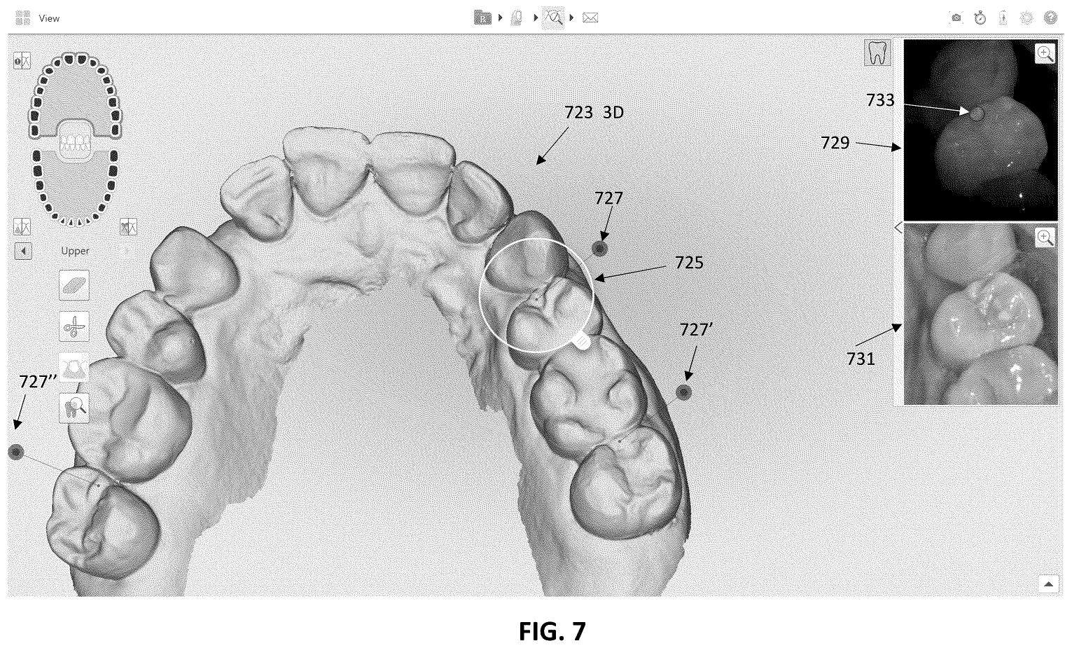

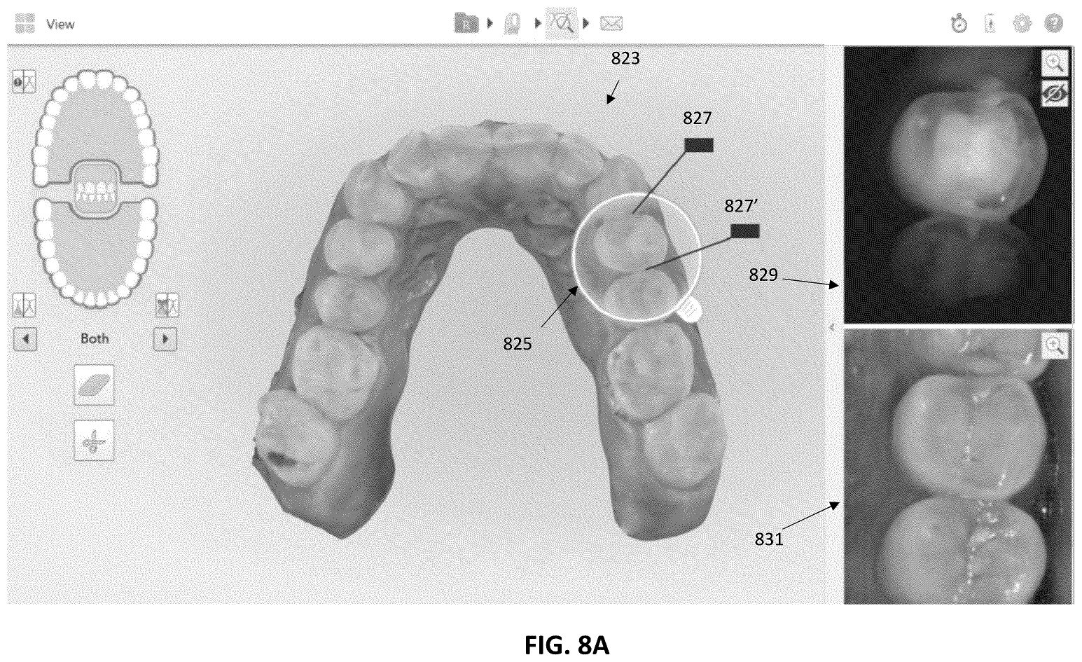

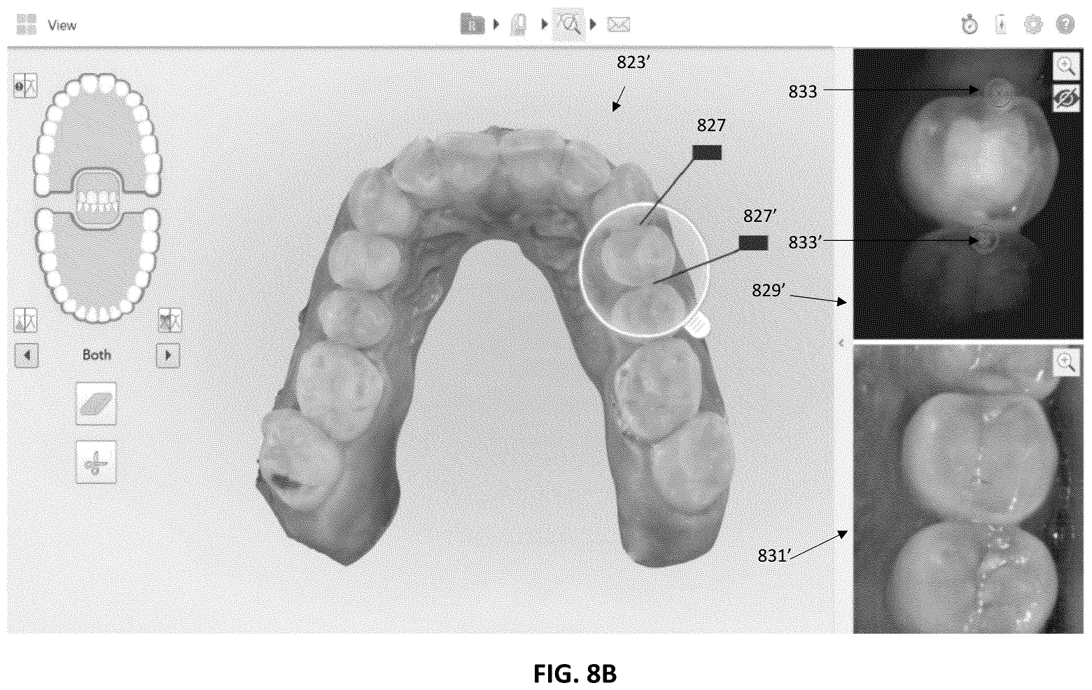

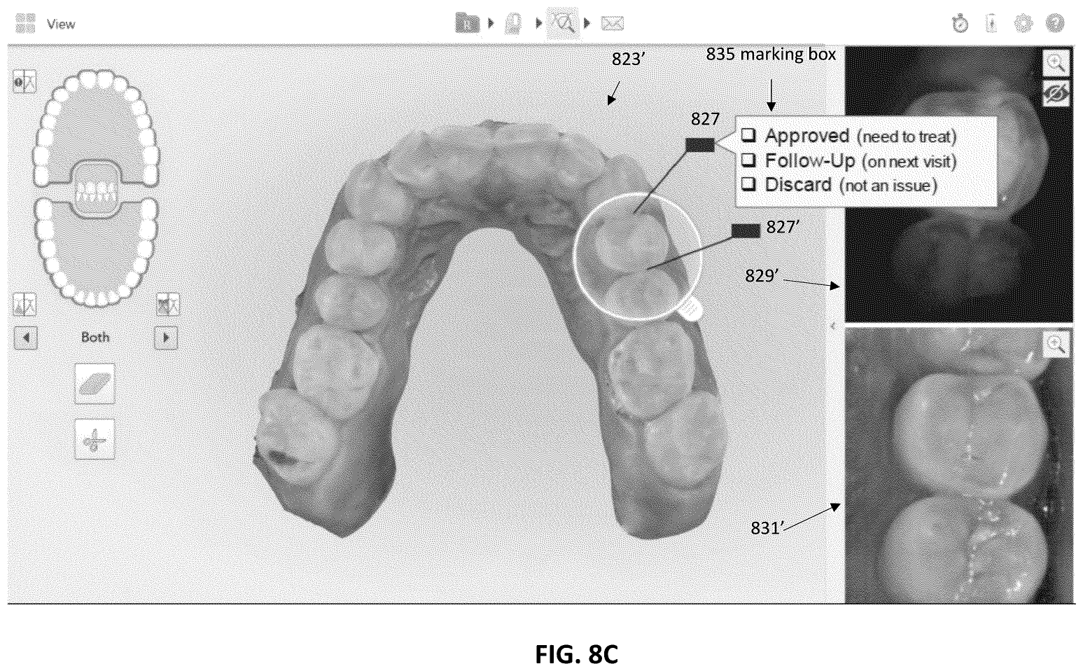

FIGS. 7 and 8A-8C described in reference to example 2 below, illustrate one example of a user interface as described herein.

### Example 1

A pattern-recognition agent was trained on a database of intraoral scan data in which both surface scanning (e.g., RGB) and near-infrared (near-IR) scan data of patient teeth were used, to generate a three-dimensional (3D) surface model of the teeth. The training data included identified cavities of various sizes and depths. A system as described herein was used with the trained pattern-recognition agent. Test intraoral scan data included four channels corresponding to each image for each of a plurality of dental images that had been taken using an intraoral scanner were provided to the trained pattern recognition agent. For each image, the trained pattern recognition agent zero, one or more possible caries center points. The caries center points corresponded to putative locations from each image representing a center of a suspected caries in the images. In this example, the four channels used include: a near-IR channel for near infrared image, and 3 visible light (RGB) channels. The detected caries centers where then projected onto the 3D dental surface, which was either constructed from the scan data after scanning or was constructed in real-time with the scanning. Each of the detected centers were projected onto the 3D image. The projected centers were then aggregated/combined to give the final detections. Not all image detections yield surface detections.

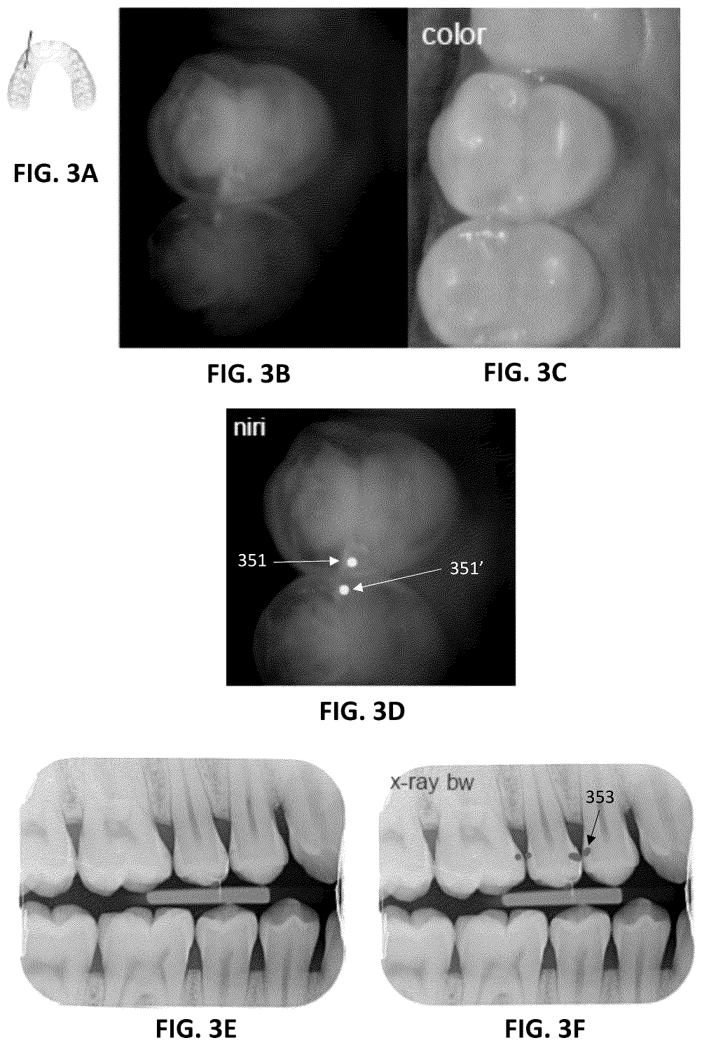

For example, FIGS. 3A-3F illustrate one example of caries prediction using a method as described above and illustrated schematically in FIG. 2. The jaw (FIG. 3A) was scanned using an intraoral scanner, which scanned for both color (red, green, blue) and near-IR. The scan was used to generate a virtual 3D model of the teeth, and a subset of the scanned image were analyzed to detect caries. FIG. 3B shows an example of a near-IR channel from the subset of examined scans. FIG. 3C shows a color channel (combining red, green and blue) of the corresponding image. A trained pattern-matching agent was applied to each image (including the four channels) and the resulting data for these images was combined. In this example the agent was trained using a training data set of ˜300 patients, ˜60K images (including both near-IR and color channels), having ˜2 caries per case. A set of approximately 2.5K images were examined from the scan data for the test patient shown in FIGS. 3A-3F. As shown in FIG. 3D, caries were identified. These putative caries were confirmed by aggregating (using a voting technique as described above) a plurality of different images of the same region of the teeth, and projecting onto the 3D virtual model. Segmentation indicated that at least two adjacent caries regions were present between the two teeth shown. In FIG. 3D, the two confirmed caries were labeled on the near-IR image by marking the caries centers 351, 351′. The final results were compared to results from dental x-rays of the same teeth, as shown in FIGS. 3E and 3F. As shown in FIG. 3F the comparison with the x-ray data indicated the presence of caries, confirming the automatic caries detection results.

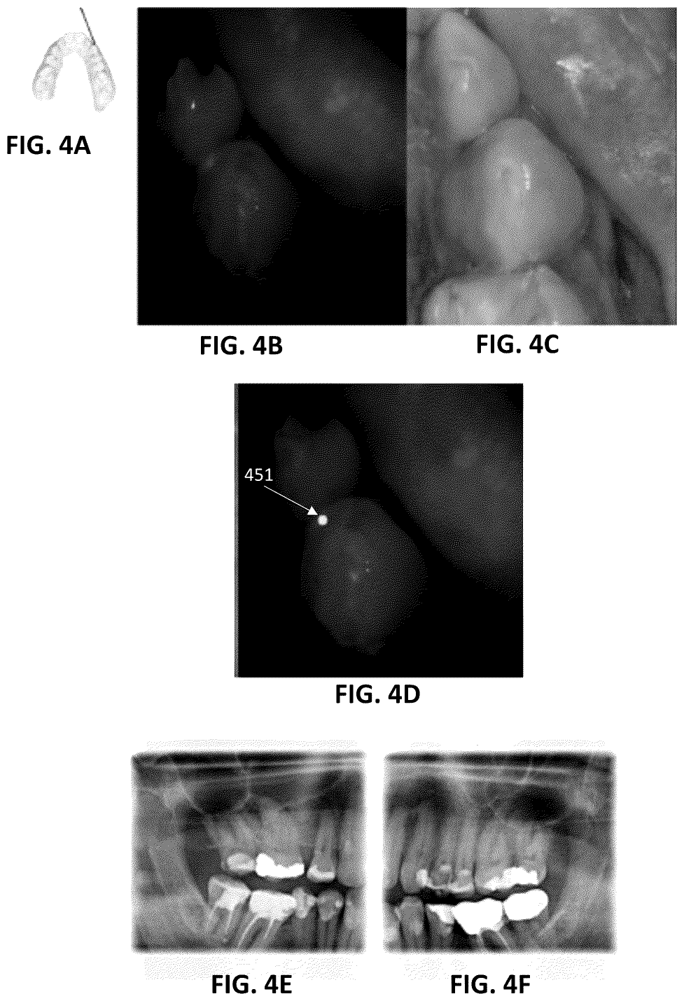

FIGS. 4A-4F illustrate another example of the successful prediction of a caries based on scan data. A 3D virtual surface model of the teeth is shown in FIG. 4A. FIG. 4B shows an example of a near-IR channel from the subset of examined scans. FIG. 4C shows a color channel (combining red, green and blue) of the corresponding image. After completing the automatic caries recognition method described above, a caries was detected at a sufficiently high confidence level, e.g., above the threshold determined form a test data set so that the likelihood that a putative carries is real, after aggregating a plurality of overlapping regions taken at different camera angles is 30% or greater (e.g., 35% or greater, 40% or greater, 45% or greater, 50% or greater, 55% or greater, 60% or greater, 65% or greater, 70% or greater, 75% or greater, 80% or greater, 85% or greater, 90% or greater, etc.). The caries center 451 corresponding to this caries is shown in the representative image of FIG. 4D. This identification was in keeping with the results from corresponding x-ray data, as shown in FIGS. 4E-4F.

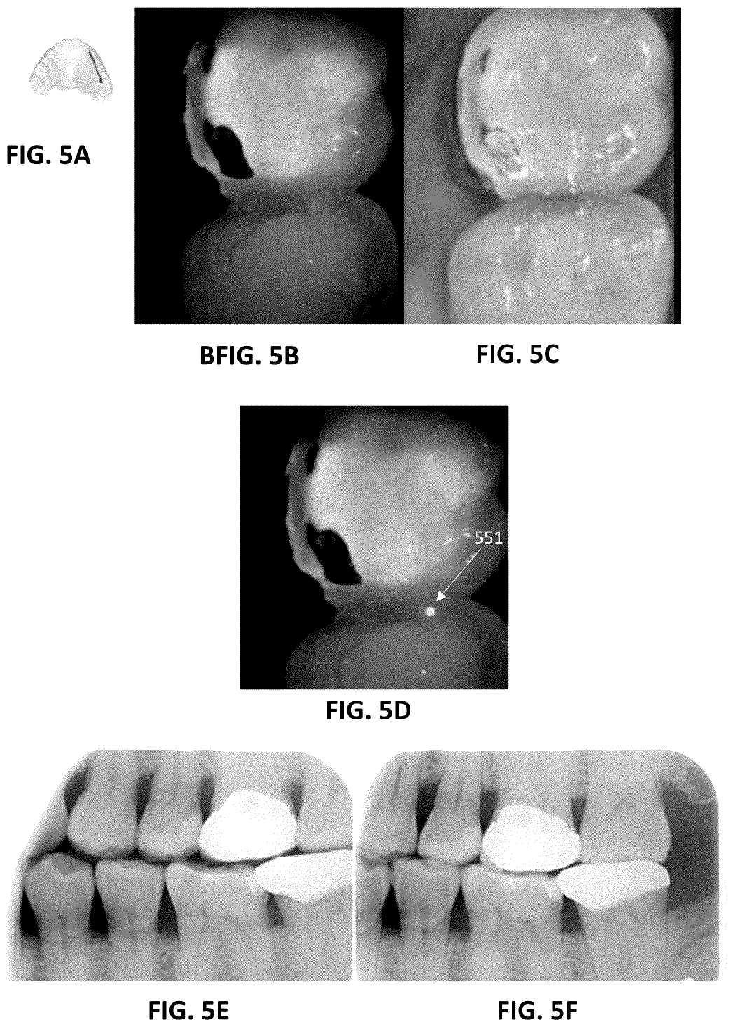

Similar results were seen in teeth including existing dental work, including metallic fillings. This is illustrated in the examples shown in FIGS. 5A-5F. A 3D virtual surface model of the teeth is shown in FIG. 5A. FIG. 5B is an example of a near-IR channel from the subset of examined scans. FIG. 5C shows a color channel (combining red, green and blue) of the corresponding image. After completing the automatic caries recognition method described above, a caries was detected at a sufficiently high confidence level, e.g., above the threshold determined form a test data set so that the likelihood that a putative carries is real, after aggregating a plurality of overlapping regions taken at different camera angles. The caries center 551 corresponding to this caries is shown in the representative image of FIG. 5D. This identification was in keeping with the results from corresponding x-ray data, as shown in FIGS. 5E-5F.

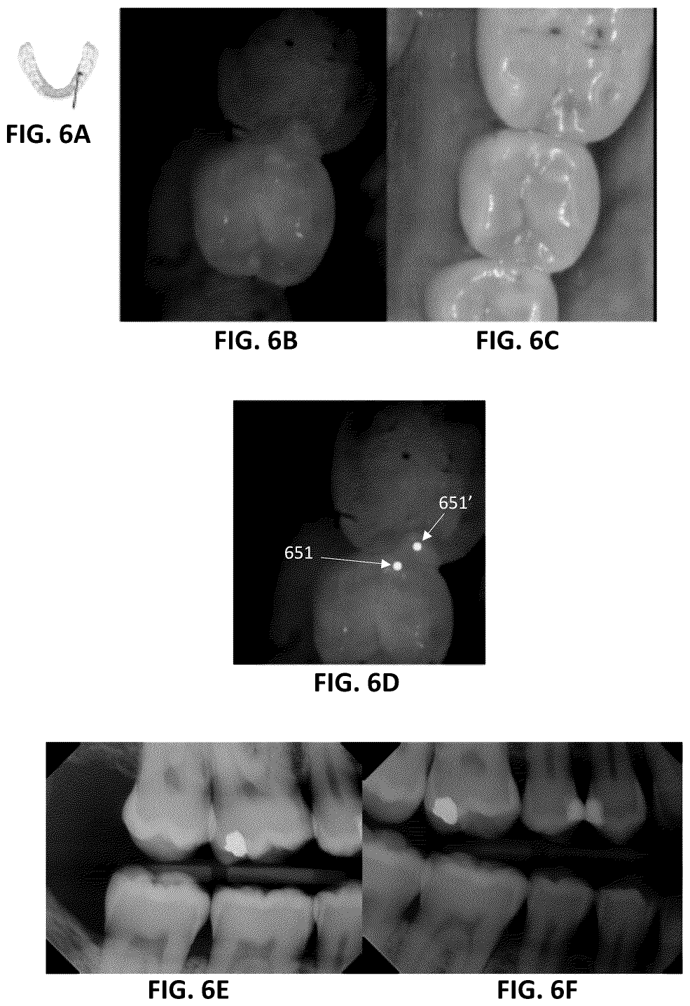

Similar results are shown in another example having multiple caries, shown in FIGS. 6A-6F. The intraoral scan data was used to generate a virtual 3D model of the teeth (shown in FIG. 6A), and a subset of the scanned image were analyzed to detect caries. FIG. 6B shows an example of a near-IR channel from the subset of examined scans. FIG. 6C shows a color channel (combining red, green and blue) of the corresponding image. A trained pattern-matching agent was used to determine putative caries (and corresponding caries centers, caries boundaries, etc.). As shown in FIG. 6D, two caries were identified between two adjacent teeth (within the interproximal region. These putative caries were confirmed by aggregating (using a voting technique as described above) result from the trained pattern recognition agent data for each of a plurality of different images of the same region of the teeth, and projecting onto the 3D virtual model. Segmentation of the 3D model indicated that the caries regions were present on two separate teeth. In FIG. 6D, the two confirmed caries were labeled on the near-IR image by marking the caries centers 651, 651′. The final results were compared to results from dental x-rays of the same teeth, as shown in FIGS. 6E and 6F, confirming the automatic caries detection results.

As mentioned, an intraoral scanner may be configured to detect caries as the scanner is operating (e.g., in real time or near-real time), for example as the scanner is scanning the teeth. During operation, the apparatus may build the virtual 3D model of the teeth and may (in real or near-real time) project putative or confirmed caries indicators (e.g., caries centers) from the 2D image onto the virtual 3D model. For example, the apparatus may aggregate the putative caries (e.g., caries centers, caries boundaries, etc.) and may indicate as points on the virtual 3D model of the teeth and/or one or more 2D images. A single detection flag may be included on the 3D model and/or 2D image that may be selected (e.g., by clicking on) to indicate the presence of a likely caries. The use of a caries center or point on the 3D model/2D image(s) may allow the user to see the likely caries directly. Thus, a ‘best’ or representative image may be used when displaying the 2D image(s). The apparatus may be configured to allow the user to select (e.g. “click on”) the flag (corresponding to the carries center) to see data about the caries and/or the most relevant image indicating or suggesting the dental caries. In some cases, this may be done using a review tool looking at the virtual 3D model and/or the 2D images (e.g., a near-IR review tool). In general, these methods and apparatuses may show the detection points in the 2D images in the NIRI review tool.

### Example 2

In some examples, the caries detection was implemented as part of an intraoral scanner in real time as the scanner was being operated. The detection neural network was implemented as part of the intraoral scanner workflow, and included: running inference on images while scanning, in real time; projecting detected caries center points from the scanned 2D images onto constructed 3D model generated from the 2D (surface) scanning images; aggregating 3D points (caries centers) on the model that were sufficiently close, to result in a single 3D detection flag, e.g., an aggregate caries center; enabling the user to click a flag to see the most relevant image with detection in the NIRI review tool; and showing the detection points in the 2D images in the NIRI review tool.

For example, FIG. 7 illustrates one example of a user interface that may be used to review automatically detected caries. FIG. 7 illustrates one example of a user interface illustrating the caries detection as described herein. In FIG. 7 a virtual 3D model of the patient's teeth (lower jaw 723) is shown. The model may be moved, e.g., rotated, to show different views. A viewing window 725 is also shown over the model and may be moved and repositioned relative to the 3D model. Images corresponding to the region of the teeth under the viewing window, including a near-IR channel image 733 and a visible light image 731, are shown on the right of the screen. These images may be taken from the scan images directly or may be synthesized from the scan images. The images may be updated as the viewing window is moved relative to the 3D model. In FIG. 7 the 3D model 723 is marked to show the locations of three caries centers 727, 727′, 727″. In this example, either or both of the channel images 731, 733 may also include a marker for the caries centers (or a “flag” corresponding to the caries centers).

FIGS. 8A-8C shows another example of a user interface showing a virtual 3D model of a patient's teeth 823, 823′ taken using an intraoral scanner. The scan images include at least a near-IR and a red, green, blue (RGB) channels. As in FIG. 7, the viewing window 825 may be moved relative to the 3D model to show images from the near-IR 829, 829′ and RGB channels 831, 831′. In this example two caries centers 827, 827′ are shown marked on the 3D model, and the user may select them to review additional detail about the caries, including seeing them in a view from the scan images 833, 833′, as shown in FIG. 8B.

The caries centers may be selected and/or marked by the user, as shown in FIG. 8C. In this example, each caries may be marked 835 to indicate that the caries is not an issue (or is not correct), should be followed-up on, and/or is approved for treatment. These markings may be saved with the output of the caries detection, e.g., as part of the caries data structure.

The user may choose the specific 3D detection to be shown, and/or the apparatus may identify which 2D image pair for which the detection is best seen. In some examples, the apparatus may move the viewing window of the review tool to the 2D image of highest confidence and show that image pair in the user interface.

The methods and apparatuses described herein may be used with/combined with additional information to enhance diagnosis. For example, the methods and apparatuses described herein may provide the dentist with several sources of data-driven AI clinical data. One source will be the intraoral scan with the NIRI images. Another source may be dental X-rays (if done by the dentist to this specific patient in a reasonable period between the scan to the X-ray). This data may be combined for the dentist to get an all-in-one experience for practical and easy clinical decision-making.

The sensitivity for detection of caries may be adjusted based on the availability of these additional sources of data. For example, if in a specific area, prior caries detection has not found any caries, the system may change the sensitivity for that specific area, to be sure that no caries will be missed.

The user interfaces described herein may allow a dental professional to view the entire dentition, both arches side by side, with all detections visible on the 3D models. The dental professional may be able to choose each detection and focus on it. In some cases, the apparatus or method may include a workflow tool that may take the dental professional through all the detected caries in order to ensure no detection is missed. In some cases, the dental professional may have access to a detection sensitivity control, which may be adjusted by the dental professional to show only high confidence detections of caries or detections of any confidence level. A dental professional may have the ability to define their preference for default sensitivity when opening a case for review. In some cases, the user interface may be configured to allow the user (e.g., dental professional) to tag the detected caries, as described above, e.g., whether the caries is planned to be treated, or planned for follow-up, or wrong. For example, when marked for follow-up, on a subsequent visit by patient, the detected caries will be shown so user can check again that spot.

### Identifying Images Most Likely to Include Caries

As mentioned, also described herein are methods and apparatuses (e.g., intraoral scanners) that are configured to identify which images of an intraoral scan, including which near-IR and/or visible light images, are most likely to show caries. These methods and apparatuses may used to rank images of a scan, typically after the scan has been completed, though in some cases, the method may be performed in an ongoing manner, as scans are being collected. The ranking indicate the likelihood of an image (e.g., a 2d image from the scan) showing a caries even without having to actually identify caries in the image(s). The resulting rankings may be used to display images for manual review, e.g., by a user, and/or for automatic detection of caries, as described above.

Near-IR images, in particular, may be difficult to interpret given the complexity of the way in which light traverses the interior of the tooth, resulting in different signal behaviors for near-IR images. As a result, caries may be visible in some images but not readily visible in other images of the same tooth surface. Surprisingly, the ability to visualize caries in near-IR images may be predicted based on properties of the tooth (tooth surface) and imaging system (e.g., camera and illumination source) and the relationship between the two. The methods and apparatuses described herein may therefore score or rank images from a scan (e.g., an intraoral scan) based on these properties in order to identify a subset of images form the intraoral scan that are most likely to show visible caries (if the tooth being imaged actually contains a caries). Thus, some near-IR images are more likely to allow caries to be seen in the image than others. In one example, the near-IR images from a plurality of near-IR images may be processed as described herein in order to determine a likelihood that a caries would be visible from that particular near-IR image; this prediction may be based on one or more features describing a relationship between one or more reference points in an interproximal region of a tooth in the near-IR image and one or more camera parameters of a camera that is used to take the near-IR image. This may be achieved by identifying, for each image of the plurality of near-IR images, one or more reference points in an interproximal region of a tooth, and for each one or more reference point, identifying a normal to the reference point relative to the tooth, and a camera angle of the camera taking the near-IR images, and estimating an angle between the normal and the camera angle. In other examples, the accuracy of the prediction may be increased by increasing the parameters or properties used to score the image, beyond just the angle between the normal and the camera angle, as will be described in greater detail herein. In this first example, the property that is used to determine a score of how likely a carious lesion (e.g., caries) will be visible in the near-IR image may be based on the camera angle relative to a surface normal of the tooth.

Because there can be hundreds, or even thousands of near-infrared (near-IR) images taken as part of a single scan, it would be extremely helpful to select a subset of images that are most likely to show caries based on a likelihood score, as described herein. The scored images may be used to determine which images should be displayed to the user (e.g., doctor, dentist, dental professional, technician, etc.) and/or which subset of images should be used in any of the caries identification algorithms described herein. Thus, images having a sufficiently high score may be shown to the user only after this scoring/selection process. The score may be relative to the other images, or they may be independent of the other images.

Different scoring techniques may be used. For example, a first scoring technique may be based on tooth orientation in relation to the near-IR light source and/or camera position, as mentioned above. Alternatively, or additionally, the scoring may be instead based on a trained machine learning agent that uses multiple inputs or properties which may be taken or measured from the image(s). For example, a machine learning agent trained as a classifier may be used by training on properties such as geometrical features of the tooth/tooth surfaces and grey level information from the near-IR images.

In general, there methods and apparatuses may be configured to grade the near-IR images to determine a likelihood that these images will show visible caries in the near-IR image. In cases where the images having a high likelihood of showing a caries do not include a caries, this may provide a useful negative control for the user. In general, grading the near-IR images with a likelihood for each image that it will show caries (or will develop into caries) may be more efficient, e.g., more quickly performed, and may be performed as part of the apparatus requiring less time and computational power than analyzing these images to identify actual or potential caries. Further, even in cases in which caries may be present, it may be difficult to see the caries in some images, given the lighting/position of the caries. Thus, these methods may be performed in combination with any of the caries detection techniques discussed above, in order to show to the user the best image (e.g., the images most likely to include a visible caries. In some cases, the score/grading mechanism may be used as part of an entirely local process that may be part of the intraoral scanner itself, and may not require a remote processor.

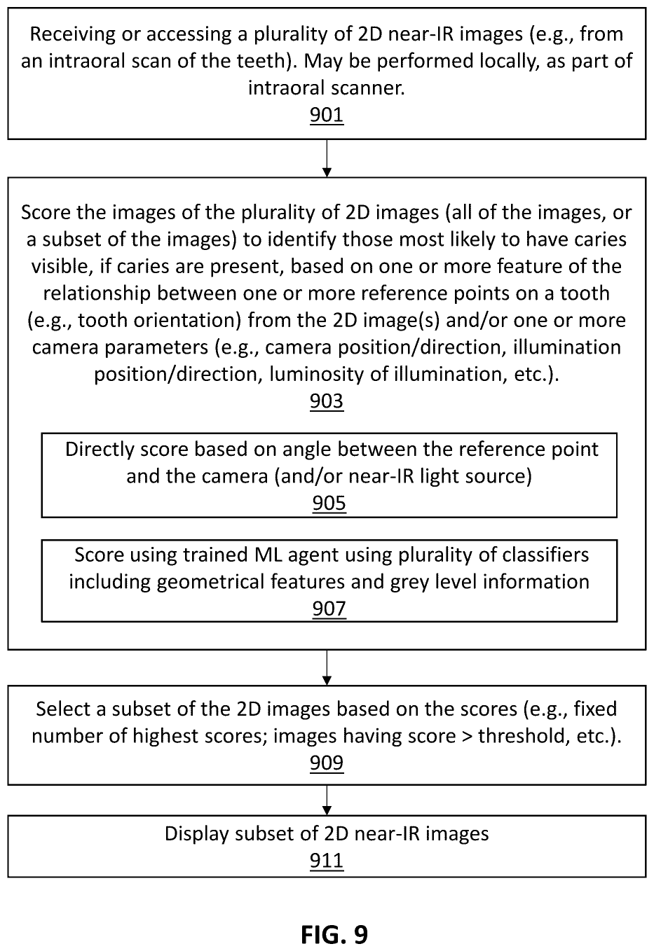

For example, FIG. 9 illustrates a schematic example of a method of identifying near-IR images that are most likely to show a caries. As shown in FIG. 9, The method may include receiving (and/or accessing) a plurality of 2D near-IR images, e.g., from an intraoral scan of the subject's dentition. 901 The processor(s) performing this method may be part of the intraoral scanner (e.g., may be local). As mentioned, because this technique may be performed quickly and requires low computational power, particularly as compared with methods requiring identification of actual caries, it may be readily performed as part of the intraoral scanner.

The images may then be scored 903. All or a subset of the images of the plurality of 2D images may be scored to identify those most likely to have caries visible, if caries are present. Scoring may be relative to an absolute scale (e.g., regardless of the scores of the other images) or may be relative to the other images from the scan. In some cases, the scores may be normalized to the other images. In general, scoring may be based on one or more features of the relationship between one or more reference points on a tooth (e.g., tooth orientation) from the 2D image(s) and/or one or more camera parameters (e.g., camera position/direction, illumination position/direction, luminosity of illumination, etc.). The images to be scored may be the original images, or they may be modified from the original scan (e.g., filtered, smoothed, interpolated, etc.). The images may be near-IR images. In some cases, the images may be or may include color images, fluorescent images, or other imaging types, or combinations of these (e.g., an image showing both visible light and near-IR, for example).

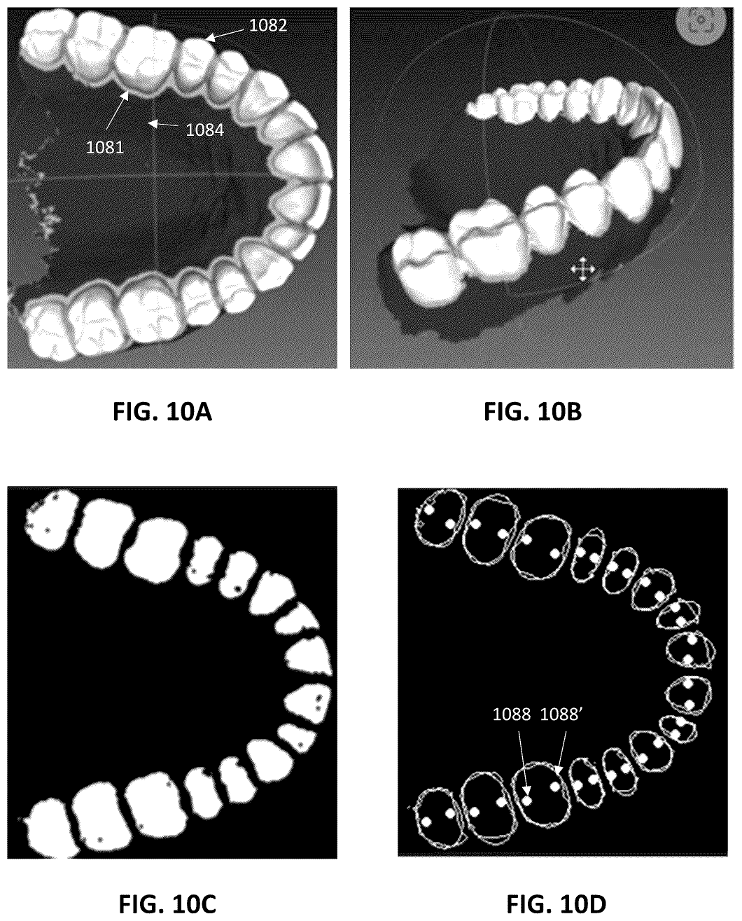

Prior to scoring, the images may be prepared. For example, as each image is scored, it may be analyzed, filtered, etc. In some cases, the images may be first examined to identify the one or more reference points, e.g., in the interproximal region. For example, one or more reference points may be identified on each tooth, such as a reference point from the distal (e.g., distal interproximal) interproximal region and/or a reference point from the medial (e.g., medial interproximal) region of each tooth. The reference points may be identified using the 3D model of the teeth (e.g., digital model, such as a digital surface model of the subject's dentition). The one or more reference points may be mapped (in some cases from the 3D digital model) to the 2D image(s), such as the near-IR images. FIGS. 10A-10D, discussed in detail below, show one example of a method of identifying one or more reference points.

In some cases, scoring may be performed by determining, for each of the one or more reference points present in the 2D image, using one particular feature that is well-correlated with the presence and/or absence of dental caries. For example, scoring may include directly scoring the images based on angle between the reference point and the camera (and/or near-IR light source) 905. In this case, the feature is the relationship, e.g., the angle, between a normal on the tooth surface at the reference point, providing tooth orientation, and the camera angle (e.g., a camera parameters that includes the direction of the camera).

Alternatively, in some cases, multiple different parameters may be used, including some that relate more specifically to the relationship between the tooth (tooth orientation) and the imaging system (e.g., camera, lighting, etc.) or relate to the imaging system (e.g., relationship between the camera and the light source(s)), and/or the tooth. These multiple parameters may be used as classifiers for use with a trained machine learning agent (e.g., algorithm) 907. For example, a trained ML agent may use a plurality of classifiers including geometrical features and grey level information to determine a score. Any number of different classifiers may be used. As a non-limiting example, the classifiers that may be used may include one or more of: cosine of the angle between camera and normal at reference point, Z absolute value of reference point to camera vector; camera to reference point distance in mm; cosine of the angle between camera direction and reference point; absolute of z value of camera direction vector; cosine of the angle between camera refraction and tooth axis; absolute z value of light source (e.g., LED) direction vector; cosine of the angle between a first light source (e.g., LED1) and normal; cosine of the angle between LED1 direction and reference point; distance between LED1 location and reference point in mm; distance between LED1 location and camera in mm; LED1 luminosity; absolute of z value of reference point to LED1 vector; cosine of the angle between a second light source (e.g., LED2) direction and normal; LED2 luminosity; absolute of z value of reference point to LED2 vector; cosine of the angle between LED2 direction and reference point; distance between LED2 location and reference point in mm; and/or distance between LED2 location and camera in mm.

Once scored, the scores may be adjusted (e.g., normalized) and may be used to filter or otherwise sort the images and may be used to generate one or more sub-sets of images. For example, these methods may select a subset of the 2D images based on the scores. The higher scores may correspond to a greater likelihood of seeing a dental caries on the image. In general, the higher scoring images may be displayed to the user (e.g., doctor, clinician, dentist, orthodontist, etc.). The higher scoring images may be selected for display and/or saving (e.g., as a subset of the total images in the scan) 909. In some cases, those images having a score that is greater than a minimum threshold may be displayed. If scores as between 0 and 1.0 or any other range, the threshold may be adjusted so that only the highest x percent (e.g., 90%, 95%, 96%, 97%, 98%, 99%, 99.1%, etc.) are shown or selected for display. In some cases, the threshold may be set and/or adjusted by the user (e.g., in a user display/interface, the user may select, or adjust, this threshold up/down). In some cases, the method or apparatus may simply display the top y number of most likely e.g., a fixed number of highest scores; images having score>threshold, etc.). Alternatively, or additionally, the higher-scoring images may be displayed 911. Thus, in any of these examples a separate selection step, 909, is not required, but instead the scored images may be directly displayed, e.g., in a user interface.

The scores may be stored (e.g., in a data field, as metadata, etc.) and/or an index of the scores including the corresponding images or reference to uniquely identify the images, may be stored locally and securely by or within the intraoral scanner and/or a remote server. As mentioned here, any appropriate user interface and display may be used, including showing the high-scoring images individually or as a group or groups. The user may switch between different images, manually or automatically. In some cases, the user may select region or teeth (e.g., on a 3D model/display, including a 3D surface model) and be shown a 2D image having a high score that corresponds to the selected tooth or teeth (ore region). The 2D image may include markings indicating the presence of a high-scoring 2D image that is or can be, displayed. In some cases, images that do not have a sufficiently high score (e.g., below a threshold) may not be shown, and/or may be “discarded” from the user interface. Alternatively, in some cases, the user may select to specifically be shown some or all of the lower-scoring images.

As mentioned, in some cases, the method or apparatus may use the angle between a representative point and the camera and/or light source as a parameter, either on its own or as a parameter input into a trained machine learning agent. For example, the method or apparatus may be configured to compute a score that is based on the angle between a normal to the surface of the tooth at the selected representative tooth and the primary transmission angle of a light source emitting the light used to capture the image (e.g., one or more LED that is used for transmitting the near-IR light to the teeth). In some cases, multiple light sources may be used. The location of the light from the light source and the position of light in relation to the possible carious lesion may impact the visibility of the caries. Thus, the score may be implemented based on an angle that is formed between the normal and the source of light from the LED; for example, the smaller the angle, the higher the score. In cases where very close or near images are scored similarly high, near-duplicate or very similar and/or overlapping images may be removed or marked as duplicate (and may not need to be displayed). In some cases, only on high-scoring image (e.g., highest scoring image) may be selected and/or displayed.

As mentioned, any of these methods and apparatuses may include determining a representative or reference point, also referred to as an anchor point, that may be used as a basis of calculation. Since the location of an actual carious lesion is not known, these reference points may be broadly selected as areas where the carious lesion may be, for example, within the interproximal (IP) region of a tooth. This reference point may be used to determine a normal (surface normal) from the tooth.

FIGS. 10A-10D illustrate one example of a technique for identifying the reference point(s) on each tooth from the scan. As mentioned, a medial and proximal point may be determined. The method and/or apparatus may estimate (e.g., calculate, approximate, etc.) the interproximal region from a segmented image or model (e.g., 3D model) of the teeth. FIGS. 10A and 10B show a 3D digital model of the dentition that has been segmented into teeth 1082 and gingiva 1084. The interproximal region 1081 is highlighted. Segmentation may identify these different regions and parts. If a 3D model is used to identify these points the corresponding regions on the 2D images (near-IR images) may be identified and used.

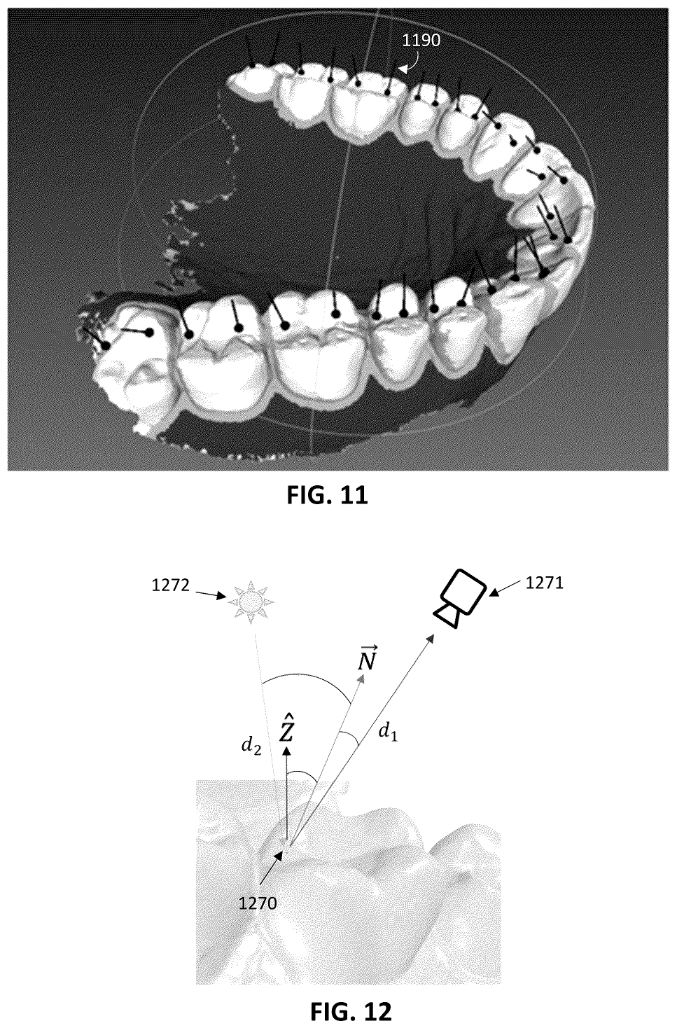

The segmented surface images/models shown in FIGS. 10A-10B may be used to identify interproximal regions, as shown in FIG. 10C, and from these images/models reference points 1088, 1088′ (two for each tooth) may be determined, as shown in FIG. 10D, and used so that for each image, e.g., each near-IR image, in the scan, the reference points may be used in to determine the orientation of the tooth or tooth surface, which can be used to determine one or more property. For example, in FIG. 11, an average normal 1190 to the tooth surface may be determined around each point. This normal may be used to determine the single classifier (e.g., the angle between the camera angle and/or light source) and/or the some of the multiple classifiers that may be used to determine the score as described above. For example, in one non-limiting example, the score may be based on a trained ML agent that uses 19 geometrical features as classifiers that describe the relationships between the camera, LEDs, and interproximal reference points. These classifiers may include refraction, the relative angles between the camera angle, light source(s), and the tooth surface, LED luminosity, etc. For example, refraction (e.g., refraction of a camera ray to IP reference point) may be used. In some cases, LED luminosity, e.g., calculated as fraction of light emitted in the IP point direction compared to portion of light that is projected directly, may be used.

FIG. 12 illustrates some of the relationships between the teeth (e.g., representative point 1270), camera 1271, a light source 1272.

In practice these methods and apparatuses for predicting images that may best show a carious lesion (caries) may be used before actual caries are detected. This may provide an overall screen that may indicate (e.g., based on the number of images above threshold) the likelihood that caries are present. This may also be used to assist in manually or automatically detecting caries, e.g., using image above a predetermined threshold for the score. For example, these methods may be performed post-processing. The actual scores (e.g., numeric scores) may or may not be displayed, and may dramatically simplify and shorten the review process for images, and in particular near-IR images. In some examples, a typical scan including near-IR images may record over a thousand images (e.g., between 3-6 thousand images). These techniques may determine the small subset of such images that are likely to show caries from these near-IR images, which may be much less than 1% of the total images collected.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments, one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively, or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method, the method comprising:
receiving or collecting a plurality of two-dimensional (2D) intraoral scanner images of a patient's teeth, wherein each image comprises two or more channels, including a near-infrared (near-IR) and a visible light channel;
identifying, for each image of the plurality of 2D intraoral scanner images, a caries center for any carries in the images using a trained pattern matching agent that is trained to use the two or more channels of each image;
projecting the caries center for any carries identified in each image onto a three-dimensional (3D) model of the patient's teeth, wherein the 3D model of the patient's teeth is generated from the plurality of 2D intraoral scanner images;
determining a consensus caries center for each of one or more caries based on the projected detected caries; and
outputting an indicator of one or more caries based on the consensus caries centers.

2. The method of claim 1, wherein identifying the caries center for any carries in the images comprises identifying caries centers and caries boundaries from each image.

3. The method of claim 1, wherein identifying the caries center for any carries in the images comprises identifying caries centers from at least 10 images of the same region of a tooth taken with different camera angles.

4. The method of claim 1, wherein determining the consensus caries center comprises aggregating the projected caries centers into groups of projected caries centers, by applying a distance threshold to the projected caries.

5. The method of claim 1, wherein outputting comprises outputting a caries detection 3D model of the patient's teeth including the consensus projected carries centers distributed on the caries detection 3D model.

6. The method of claim 1, wherein outputting comprises outputting a caries data structure.

7. The method of claim 1, further comprising segmenting the 3D model of the patient's teeth before or after projecting the detected caries center to the 3D model.

8. The method of claim 1, wherein identifying the caries center for any carries in the images further comprises confirming that the caries center corresponds to a carries based on a comparison of a subset of images of the plurality of 2D intraoral scanner images having an overlapping view of a region of a tooth including the caries center, wherein each of the plurality of images is taken from a different camera angle.

9. The method of claim 8, wherein the subset of images of the plurality of 2D intraoral scanner images having the overlapping view of the region of the tooth including the caries center comprises at least 3 images.

10. The method of claim 8, wherein confirming the caries center comprises applying a threshold based on a number and/or a caries confidence score for each image of the subset of images.

11. The method of claim 1, wherein receiving or collecting the plurality of 2D intraoral scanner images of the patient's teeth comprises receiving the plurality of 2D intraoral scanner images from an intraoral scanner data set.

12. The method of claim 1, wherein receiving or collecting the plurality of 2D intraoral scanner images of the patient's teeth comprises receiving a plurality of 2D image comprising four or more channels including a near-infrared (near-IR), a red light, a green light, and a blue light channel.

13. The method of claim 1, wherein identifying the caries center for any carries in the images using the trained pattern matching agent comprises identifying caries centers for each of at least 3 images having overlapping tooth regions of the plurality of 2D intraoral scanner images.

14. The method of claim 1, wherein determining the consensus caries center for each of one or more caries based on the projected detected caries comprises generating a caries data structure including coordinates of each of the consensus caries center, a severity of the caries, a reference to a representative image for each caries.

15. A method, the method comprising:
receiving or collecting a plurality of two-dimensional (2D) intraoral scanner images of a patient's teeth, wherein each image comprises two or more channels, including a near-infrared (near-IR) and a visible light channel;
identifying, for each image of the plurality of 2D intraoral scanner images, a caries center for any carries in the images using a trained pattern matching agent that is trained to use the two or more channels of each image;
confirming that the caries center corresponds to a carries based on a comparison of a subset of images of the plurality of 2D intraoral scanner images having an overlapping view of a region of a tooth including the caries center, wherein each of the plurality of images is taken from a different camera angle;
projecting the confirmed caries centers onto a three-dimensional model of the patient's teeth;
aggregating the projected caries centers to determine a consensus caries center for each of one or more caries based on the projected confirmed caries centers; and
outputting an indicator of one or more caries based on the consensus caries centers.

16. An intraoral scanning system, the system comprising:
a wand comprising a near-infrared (near-IR) light source and one or more cameras;
one or more processors; and
a memory coupled to the one or more processors, the memory storing computer-program instructions, that, when executed by the one or more processors, cause the processor to:
access a plurality of near-infrared (near-IR) images of a subject's teeth taken by the wand;
score each near-IR image of the plurality of near-IR images to provide on a likelihood that a caries is visible in the near-IR image based on one or more features describing a relationship between one or more reference points in an interproximal region of a tooth in the near-IR image and one or more camera parameters of a camera taking the near-IR image by: identifying, for each image of the plurality of near-IR images, the one or more reference points, a normal to the reference point relative to the tooth, and a camera angle of the camera taking the near-IR images, and estimating an angle between the normal and the camera angle; and
display near-IR images from the plurality of near-IR images based on the scores.

17. The intraoral scanning system of claim 16, further comprising a base enclosing the one or more processors and the memory.

18. The intraoral scanning system of claim 16, wherein the wand further comprises one or more visible light sources.

19. The intraoral scanning system of claim 16, wherein the instructions cause the one or more processors to score near-IR images having smaller angles between the normal and a camera angle higher than images having larger angles between the normal and the camera angle.

20. The intraoral scanning system of claim 16, wherein the instructions cause the one or more processors to identify, for each image of the plurality of near-IR images, one or more of: a light source vector for each of one or more light sources, vector between the reference point and the camera, a tooth axis for the tooth, the angle between the tooth axis and the camera angle, a luminosity of each of the one or more light sources, a refraction of the camera angle to the reference point, distances between the camera and the reference point, distances between the camera and the light sources, distance between the camera and the one or more light sources.

21. The intraoral scanning system of claim 16, wherein the instructions cause the one or more processors to score comprises using a trained machine-learning (ML) agent to score based on the one or more features.

22. The intraoral scanning system of claim 21, wherein the one or more features comprises one or more of: a function of an angle between the camera and a normal at the reference point, a measure the z distance of a vector between the reference point and the camera, a function of an angle between the camera direction and the reference point, a magnitude of a camera direction vector, a function of an angle between the camera refraction and a tooth axis of the tooth, a magnitude of a light source direction vector, a function of an angle between a light source direction vector and the normal at the reference point, a function of an angle between the first light source direction vector and the reference point, a distance between the first light source direction and the reference point, a distance between the first light source and the camera, a luminosity of the first light source, a measure of the z-distance of a vector between the reference point and the first light, a function of an angle between a second light source direction vector and the normal at the reference point, a luminosity of the second light source, a z-magnitude of the vector between the reference point and the second light source, a function of an angle between the direction of the second light source and the reference point, a distance between the second light source and the reference point, and a distance between the second light source and the camera.

23. The intraoral scanning system of claim 16, wherein the instructions cause the one or more processors to score by identifying the one or more reference points on the interproximal region of each tooth by segmenting the plurality of 2D images to identify a tooth surface, identifying the interproximal region, and selecting the one or more reference points from within the interproximal region.

24. The intraoral scanning system of claim 16, wherein the instructions cause the one or more processors to score each image of the plurality of near-IR images by generating a score between 0-1.

25. The intraoral scanning system of claim 16, wherein the instructions cause the one or more processors to by associating the score with each near-IR image in a database including the near-IR images.

26. The intraoral scanning system of claim 16, wherein the instructions cause the one or more processors to select near-IR images from the plurality of near-IR images for display having a score that is more than or equal to a threshold.

27. The intraoral scanning system of claim 16, wherein the instructions cause the one or more processors to rank the near-IR images from the plurality of near-IR images by score and display a predefined number of near-IR images having the highest ranking scores.
